(19) United States
(12) Patent Application Publication
(10) Pub. No.: US 2023/0210744 A1
(43) Pub. Date: Jul. 6, 2023

(54) SEMI-PERMANENT TATTOOS

(71) Applicant: Inkbox Ink Inc., Toronto (CA)

(72) Inventors: Ingrid UM NLEND, Toronto (CA); Tyler J HANDLEY, Toronto (CA)

(73) Assignee: Inkbox Ink Inc., Toronto (CA)

(21) Appl. No.: 18/001,193

(22) PCT Filed: Jun. 9, 2021
(86) PCT No.: PCT/CA2021/050790

§ 371 Date: Dec. 8, 2022

Related U.S. Application Data
(60) Provisional application No. 63/037,381, filed on Jun. 10, 2020.

(51) Int. Cl. A61K 8/49; A61Q 1/02

(52) U.S. Cl. CPC A61K 8/498; A61Q 1/025; A61K 2800/43; A61K 2800/524

(57) ABSTRACT

The present disclosure relates to semi-permanent tattoos, for example, genipin-based ink compositions. In some embodiments, the ink composition comprises: genipin present in an amount of about 10% to about 20% w/w of the composition; a solvent present in an amount of about 65% to about 80% w/w of the composition; and one or more of a thickening agent, humectant, and film-forming agent present in an amount of 0.01% to about 10% w/w of the composition. In some embodiments, the composition further includes a preservative.

### CROSS-REFERENCE TO RELATED APPLICATIONS

This application claims priority to U.S. Provisional Application No. 63/037,381 filed on Jun. 10, 2020, which is herein incorporated by reference in its entirety.

This invention relates to semi-permanent tattoos, and more particularly to genipin-based ink compositions and the application of such compositions to skin using a pen-like applicator.

### BACKGROUND

Temporary or semi-permanent tattoo or body inks have been used throughout human history to decorate the body. Generally, temporary or semi-permanent tattoos are transferred to the skin through the direct exposure of the skin to tattoo ink over a designated incubation period. The quality of the transferred image and its duration on the skin can depend on the ink distribution profile in the outer layer of the skin called the stratum corneum. Current manufacturing processes for pre-fabricated tattoo designs include the utilization of flexographic or gravure printing, stencils, and inkjet printers. However, due to the barrier properties of the stratum corneum, many of the current methods require moderate oversight by the end user during the tattoo application process to ensure adequate ink delivery to the skin such that the tattoo ink develops properly.

### SUMMARY

Provided herein are compositions comprising: a semi-permanent colorant; a solvent; and a thickening agent. Also provided herein are compositions comprising: a semi-permanent colorant; a solvent; and a film-forming agent.

In some embodiments, the semi-permanent colorant is present in an amount of about 10% to about 20% w/w of the composition. In some embodiments, the semi-permanent colorant is present in an amount of about 12% to about 16% w/w of the composition. In some embodiments, the semi-permanent colorant is present in an amount of about 14% w/w of the composition. In some embodiments, the semi-permanent colorant is genipin.

In some embodiments, the semi-permanent colorant is present in an amount of about 5% to about 15% w/w of the composition. In some embodiments, the semi-permanent colorant is present in an amount of about 9% to about 11% w/w of the composition. In some embodiments, the semi-permanent colorant is present in an amount of about 10% w/w of the composition. In some embodiments, the semi-permanent colorant is genipin.

In some embodiments, the solvent is present in an amount of about 65% to about 80% w/w of the composition. In some embodiments, the solvent is present in an amount of about 72% to about 76% w/w of the composition. In some embodiments, the solvent is present in an amount of about 75% to about 7680 w/w of the composition. In some embodiments, the solvent is present in an amount of about 74% w/w of the composition. In some embodiments, the solvent is present in an amount of about 77% w/w of the composition. In some embodiments, the solvent comprises water, methanol, ethanol, isopropanol, diethylene glycol monoethyl ether, or a combination thereof.

In some embodiments, the solvent is a combination of water and ethanol. In some such embodiments, the water is present in an amount of about 25% to about 30% w/w of the composition. In some such embodiments, the water is present in an amount of about 28% w/w of the composition. In some such embodiments, the ethanol is present in an amount of about 40% to about 50% w/w of the composition. In some such embodiments, the ethanol is present in an amount of about 45% to about 47% w/w of the composition. In some such embodiments, the ethanol is present in an amount of about 46% w/w of the composition.

In some embodiments, the solvent is a combination of water and ethanol. In some such embodiments, the water is present in an amount of about 25% to about 35% w/w of the composition. In some such embodiments, the water is present in an amount of about 30% w/w of the composition. In some such embodiments, the ethanol is present in an amount of about 40% to about 55% w/w of the composition. In some such embodiments, the ethanol is present in an amount of about 45% to about 50% w/w of the composition. In some such embodiments, the ethanol is present in an amount of about 48% w/w of the composition.

In some embodiments, the composition further comprises a thickening agent. In some embodiments, the thickening agent is present in an amount of about 0.01% to about 0.3% w/w of the composition. In some embodiments, the thickening agent is present in an amount of about 0.05% to about 0.2% w/w of the composition. In some embodiments, the thickening agent is present in an amount of about 0.12% w/w of the composition. In some embodiments, the thickening agent comprises hydroxyethylcellulose, xanthan gum, arabinogalactan, aluminum starch octenylsuccinate, xanthan gum, sclerotium gum, tragacanth gum, pectin, gum karaya, gum arabic, agar, guar gum, carrageenan, locust bean gum, alginate, alginin, gelatin, tara gum, gum ghatti, gellan gum, konjac gum, cassia gum, spruce gum, chicle gum, dammar, curdlan gum, pullulan, hydroxypropyl cellulose, hydroxypropyl methylcellulose, ethylcellulose, soybean hemicellulose, and sodium carboxy methylcellulose, or a combination thereof.

In some embodiments, the thickening agent is a combination of hydroxyethylcellulose and xanthan gum. In some such embodiments, the hydroxyethylcellulose is present in an amount of about 0.01% to about 0.1% w/w of the composition. In some such embodiments, the hydroxyethylcellulose is present in an amount of about 0.04% to about 0.08% w/w of the composition. In some such embodiments, the hydroxyethylcellulose is present in an amount of about 0.06% w/w of the composition. In some such embodiments, the xanthan gum is present in an amount of about 0.01% to about 0.1% w/w of the composition. In some such embodiments, the xanthan gum is present in an amount of about 0.04% to about 0.08% w/w of the composition. In some such embodiments, the xanthan gum is present in an amount of about 0.06% w/w of the composition.

In some embodiments, the composition further comprises a film-forming agent. In some embodiments, the film-forming agent is present in an amount of about 0.1% to about 10% w/w of the composition. In some embodiments, the film-forming agent is present in an amount of about 5% to about 10% w/w of the composition. In some embodiments, the film-forming agent is present in an amount of about 4% to about 6% w/w of the composition. In some embodiments, the film-forming agent is present in an amount of about 6% to about 9% w/w of the composition. In some embodiments, the film-forming agent is present in an amount of about 5% w/w of the composition. In some embodiments, the film-forming agent is present in an amount of about 7.55% w/w of the composition. In some embodiments, the film-forming agent comprises a volatile silicon resin, polyvinylpyrrolidone, an acrylate, an acrylamide, a copolymer, an isododecane resin, or a combination thereof. In some embodiments, the film-forming agent comprises polymethylsilsesquioxane, trimethylsiloxysilicate, polypropylsilsesquioxane, dimethicone, cyclopentasiloxane, dimethiconol crosspolymer, polysilicone-6, polysilicone-8, polysilicone-11, polysilicone-14, acrylates copolymer, styrene/acrylates copolymer, polyvinylpyrrolidone, acrylates/C12-22 alkyl methacrylate copolymer, acrylates/polytrimethylsiloxymethacrylate copolymer, polyvinylpyrrolidone/vinyl acetate (VP/VA) copolymer, VP/dimethiconylacrylate/polycarbamyl/polyglycol ester, VP/dimethylaminoethylmethacrylate copolymer, VP/dimethylamino ethylmethacrylate/polycarbamyl polyglycol ester, VP/eicosene copolymer, VP/hexadecene copolymer, VP/methacrylamide/vinyl imidazole copolymer, VP/polycarbamyl polyglycol ester, VP/VA copolymer, polyester-1, polyester-2, polyester-3, polyester-4, polyester-5, polyester-7, polyester-8, polyester-10 or a combination thereof.

In some embodiments, the film-forming agent is a combination of polymethylsilsesquioxane and acrylates/C12-22 alkyl methacrylate copolymer. In some such embodiments, the polymethylsilsesquioxane is present in an amount of about 1% to about 5% w/w of the composition. In some such embodiments, the polymethylsilsesquioxane is present in an amount of about 2% w/w of the composition. In some such embodiments, the acrylates/C12-22 alkyl methacrylate copolymer is present in an amount of about 1% to about 5% w/w of the composition. In some such embodiments, the acrylates/C12-22 alkyl methacrylate copolymer is present in an amount of about 3% w/w of the composition.

In some embodiments, the film-forming agent is a combination of acrylates copolymer, polyvinylpyrrolidone, and styrene/acrylates copolymer. In some such embodiments, the acrylates copolymer is present in an amount of about 0.1% to about 10% w/w of the composition. In some such embodiments, the acrylates copolymer is present in an amount of about 0.1% to about 1% w/w of the composition. In some such embodiments, the acrylates copolymer is present in an amount of about 0.25% w/w of the composition. In some such embodiments, the polyvinylpyrrolidone is present in an amount of about 0.1% to about 10% w/w of the composition. In some such embodiments, the polyvinylpyrrolidone is present in an amount of about 0.5% to about 1.5% w/w of the composition. In some such embodiments, the polyvinylpyrrolidone is present in an amount of about 1% w/w of the composition. In some such embodiments, the styrene/acrylates copolymer is present in an amount of about 0.1% to about 10% w/w of the composition. In some such embodiments, the styrene/acrylates copolymer is present in an amount of about 5% to about 7% w/w of the composition. In some such embodiments, the styrene/acrylates copolymer is present in an amount of about 6% w/w of the composition.

In some embodiments, the composition further comprises a humectant. In some embodiments, the humectant is present in an amount of about 0.1% to about 10% w/w of the composition. In some embodiments, the humectant is present in an amount of about 1% to about 10% w/w of the composition. In some embodiments, the humectant is present in an amount of about 2% to about 6% w/w of the composition. In some embodiments, the humectant is present in an amount of about 0.5% to about 1.5% w/w of the composition. In some embodiments, the humectant is present in an amount of about 4% w/w of the composition. In some embodiments, the humectant is present in an amount of about 1% w/w of the composition. In some embodiments, the humectant comprises Aloe vera gel, lithium chloride, allantoin, urea, dicyanamideglycerol, lactic acid, propylene glycol, polyethylene glycol, dipropylene glycol, polypropylene glycol, polyethylene glycol and derivatives thereof, propanediol, hexylene glycol, 1,3-butylene glycol, 1,2,6-hexanetriol, ethoxylated glycerol, propoxylated glycerol, glyceryl triacetate, sorbitol, hydroxypropyl sorbitol, xylitol, maltitol, hyaluronic acid, Tremella extract, sodium lactate, sodium L-pyroglutamate urea, and pyrrolidone carboxylic acid, or a combination thereof. In some embodiments, the humectant is glycerin. In some embodiments, the humectant is propanediol.

In some embodiments, the composition further comprises a preservative. In some embodiments, the preservative is present in an amount of about 0.1% to about 5% w/w of the composition. In some embodiments, the preservative is present in an amount of about 2% to about 4% w/w of the composition. In some embodiments, the preservative is present in an amount of about 0.5% to about 1.5% w/w of the composition. In some embodiments, the preservative is present in an amount of about 3% w/w of the composition. In some embodiments, the preservative is present in an amount of about 1.05% w/w of the composition. In some embodiments, the preservative comprises ascorbic acid, an ascorbate, a palmitate, citric acid, a benzoate, a benzoic acid, a propionate, propionic acid, a sorbate, sorbic acid, a salicylic acid, a salicylate, hexa-2,4-dienoic acid, a hexa-2,4-dienoate, formaldehyde, a formaldehyde releaser, formic acid and its salts, 3-acetyl-6-methylpyran-2,4-(3H)-dione and its salts, 3,3′-dibromo-4,4′-hexamethylenedioxydibenzamidine and its salts, thiomersal, phenylmercuric salts, undec-10-enoic acid and its salts, 1,3-bis (2-ethylhexyl) hexahydro-5-methyl-5-pyrimidine, 5-bromo-5-nitro-1,3-dioxane, bronopol, 2,4-dichlorobenzyl alcohol, 1-(4-chlorophenyl)-3-(3,4-dichlorophenyl) urea, chlorocresol, chloroxylenol, 5-chloro-2-(2,4- dichlorophenoxy) phenol, N,N″-methylenebis[N′-[3-(hydroxymethyl)-2,5- dioxoimidazolidin-4-yl]urea], polyaminopropyl biguanide, methenamine, quaternium-15, climbazole, DMDM hydantoin, benzyl alcohol, 1-Hydroxy-4-methyl-6-(2,4,4-trimethylpentyl)-2-pyridon, piroctone olamine, bromochlorophene, o-cymen-5-ol, chlorophene, chloroacetaminde, methylchloroisothiazolinone, methylisothiazolinone, phenoxyisopropanol, chlorhexidine, chlorhexidine diacetate, chlorhexidine digluconate, chlorhexidine dihydrochloride, dimethyl oxazolidine, behentrimonium chloride, cetri-monium bromide, cetrimonium chloride, laurtrimonium bromide, laurtrimonium chloride, steartrimonium bromide, steartrimonium chloride, diazolidinyl urea, hexamidine, hexamidine diisethionate, hexamidine paraben, glutaral, 7-ethylbicyclooxazolidine, chlorphenesin, sodium hydroxymethylglycinate, silver chloride, benzethonium chloride, benzalkonium chloride, benzalkonium bromide, benzalkonium saccharinate, benzylhemiformal, iodopropynyl butylcarbamate, biphenyl-2-ol and its salts, pyrithionine zinc, an erythorbate, a nitrite, ethylenediaminetetraacetic acid (EDTA), butylated hydroxyanisole (BHA), butylated hydroxytoluene (BHT), capryllic acid, dilauryl thiodipropionate, erythorbic acid, gum guaiac, methylparaben, a sulfite, a bisulfite, a metabisulfite, propyl gallatepy, propylparaben, stannous chloride, sulfur dioxide, thiodipropionic acid, an isothiazoline, a paraben, phenoxyethanol, ethylhexylglycerin, a glycol, a tocopherol, 1,2-hexanediol, or a combination thereof.

In some embodiments, the preservative comprises a combination of phenoxyethanol and ethylhexylglycerin. In some such embodiments, the phenoxyethanol and ethylhexylglycerin are present in the composition at a ratio of about 9:1 phenoxyethanol:ethylhexylglycerin. In some such embodiments, the combination of phenoxyethanol and ethylhexylglycerin is present in an amount of about 0.1% to about 2% w/w of the composition. In some such embodiments, the combination of phenoxyethanol and ethylhexylglycerin is present in an amount of about 1% w/w of the composition.

In some embodiments, the preservative comprises 1,2-hexanediol. In some embodiments, the 1,2-hexanediol is present in an amount of about 1% to about 3% w/w of the composition. In some embodiments, the 1,2-hexanediol is present in an amount of about 2% w/w of the composition.

In some embodiments, wherein the preservative comprises a combination of phenoxyethanol and ethylhexylglycerin, the preservative further comprises EDTA. In some embodiments, the EDTA is present in an amount of about 0.01% to about 0.5% w/w of the composition. In some embodiments, the EDTAis present in an amount of about 0.05% w/w of the composition.

Also provided herein are methods of manufacturing an ink composition, the method comprising: (i) combining a thickening agent with a first solvent to form a first solution; (ii) combining genipin with a second solvent to form a second solution; and (iii) combining the first solution and second solution to form the ink composition.

In some embodiments, the methods described herein further comprise adding a film-forming agent, a preservative, or a combination thereof to the ink composition.

Also provided herein are methods of manufacturing an ink composition, the method comprising: (i) combining a thickening agent and a humectant with a first solvent to form a first solution; (ii) combining genipin and a first film-forming agent with a second solvent to form a second solution; and (iii) combining the first solution and second solution to form the ink composition.

In some embodiments, the methods described herein further comprise adding a second film-forming agent, a preservative, or a combination thereof to the ink composition.

In some embodiments, the first solution is incubated at a temperature of about 70° C. to about 120° C. In some embodiments, the first solution is incubated at a temperature of about 90° C. to about 95° C. In some embodiments, the first solution is stirred.

In some embodiments, the thickening agent is dissolved in the first solution prior to combining the first solution and second solution.

In some embodiments, the second solution is incubated at a temperature of about 30° C. to about 60° C. In some embodiments, the second solution is incubated at a temperature of about 40° C. to about 50° C. In some embodiments, the second solution is stirred.

In some embodiments, the first solution is cooled to a temperature of about 20° C. to about 50° C. In some embodiments, the first solution is cooled to a temperature of about 40° C. In some embodiments, the second solution is cooled to a temperature of about 20° C. to about 50° C. In some embodiments, the second solution is cooled to a temperature of about 40° C.

In some embodiments, the ink composition is stirred.

In some embodiments, the ink composition is cooled to a temperature of about 20° C. to about 30° C. In some embodiments, the ink composition is cooled to a temperature of about 25° C.

In some embodiments, the ink composition is cooled prior to adding the film-forming agent, preservative, or combination thereof to the ink composition.

In some embodiments, the thickening agent and first solvent are present in a ratio of about 1:500 to about 4:500 thickening agent:first solvent in the first solution. In some embodiments, the thickening agent and first solvent are present in a ratio of about 2:500 thickening agent:first solvent in the first solution.

In some embodiments, the first solvent comprises water, methanol, ethanol, isopropanol, diethylene glycol monoethyl ether, or a combination thereof. In some embodiments, the first solvent comprises water.

In some embodiments, the thickening agent comprises hydroxyethylcellulose, xanthan gum, arabinogalactan, aluminum starch octenylsuccinate, xanthan gum, sclerotium gum, tragacanth gum, pectin, gum karaya, gum arabic, agar, guar gum, carrageenan, locust bean gum, alginate, alginin, gelatin, tara gum, gum ghatti, gellan gum, konjac gum, cassia gum, spruce gum, chicle gum, dammar, curdlan gum, pullulan, hydroxypropyl cellulose, hydroxypropyl methylcellulose, ethylcellulose, soybean hemicellulose, and sodium carboxy methylcellulose, or a combination thereof. In some embodiments, thickening agent is a combination of hydroxyethylcellulose and xanthan gum. In some embodiments, combination of hydroxyethylcellulose and xanthan gum is present at a ratio of 1:1 hydroxyethylcellulose:xanthan gum.

In some embodiments, the thickening agent, humectant, and first solvent are present in a ratio of about 1:15:234 to about 1:62:187 thickening agent:humectant:first solvent in the first solution.

In some embodiments, the thickening agent and first solvent are present in a ratio of about 1:31:218 thickening agent:humectant:first solvent in the first solution.

In some embodiments, the humectant comprises Aloe vera gel, lithium chloride, allantoin, urea, dicyanamideglycerol, lactic acid, propylene glycol, polyethylene glycol, dipropylene glycol, polypropylene glycol, polyethylene glycol and derivatives thereof, hexylene glycol, 1,3-butylene glycol, 1,2,6-hexanetriol, ethoxylated glycerol, propoxylated glycerol, glyceryl triacetate, sorbitol, hydroxypropyl sorbitol, xylitol, maltitol, hyaluronic acid, Tremella extract, sodium lactate, sodium L-pyroglutamate urea, and pyrrolidone carboxylic acid, or a combination thereof. In some embodiments, the humectant is glycerin.

In some embodiments, the genipin and second solvent are present in a ratio of about 4:25 to about 3:5 genipin:second solvent in the second solution. In some embodiments, the genipin and second solvent are present in a ratio of about 3:10 genipin:second solvent in the second solution.

In some embodiments, the second solvent comprises water, methanol, ethanol, isopropanol, diethylene glycol monoethyl ether, or a combination thereof. In some embodiments, the second solvent comprises ethanol.

In some embodiments, the genipin, first film-forming agent, and second solvent are present in a ratio of about 57:16:177 to about 57:4:189 genipin:film-forming agent:second solvent in the second solution.

In some embodiments, the genipin, first film-forming agent, and second solvent are present in a ratio of about 57:8: 185 genipin:film-forming agent:second solvent in the second solution.

In some embodiments, the first film-forming agent comprises polymethylsilsesquioxane, trimethylsiloxysilicate, polypropylsilsesquioxane, dimethicone, cyclopentasiloxane, dimethiconol crosspolymer, polysilicone-6, polysilicone-8, polysilicone-11, polysilicone-14, acrylates/C12-22 alkyl methacrylate copolymer, acrylates/polytrimethylsiloxymethacrylate copolymer, polyvinylpyrrolidone/vinyl acetate (VP/VA) copolymer, VP/dimethiconylacrylate/polycarbamyl/polyglycol ester, VP/dimethylaminoethylmethacrylate copolymer, VP/dimethylamino ethylmethacrylate/polycarbamyl polyglycol ester, VP/eicosene copolymer, VP/hexadecene copolymer, VP/methacrylamide/vinyl imidazole copolymer, VP/polycarbamyl polyglycol ester, VP/VA copolymer, polyester-1, polyester-2, polyester-3, polyester-4, polyester-5, polyester-7, polyester-8, polyester-10 or a combination thereof. In some embodiments, the first film-forming agent is polymethylsilsesquioxane.

In some embodiments, the first solution and second solution are present in a ratio of about 1:4 to about 1:1 first solution: second solution in the ink composition.

In some embodiments, the first solution and second solution are present in a ratio of about 1:2 first solution: second solution in the ink composition.

In some embodiments, the film-forming agent is present in an amount of about 1% to about 10% w/w of the ink composition. In some embodiments, the film-forming agent is present in an amount of about 2% to about 6% w/w of the ink composition. In some embodiments, film-forming agent comprises a volatile silicon resin, polyvinylpyrrolidone, an acrylate, an acrylamide, a copolymer, an isododecane resin, or a combination thereof. In some embodiments, the film-forming agent comprises polymethylsilsesquioxane, trimethylsiloxysilicate, polypropylsilsesquioxane, dimethicone, cyclopentasiloxane, dimethiconol crosspolymer, polysilicone-6, polysilicone-8, polysilicone-11, polysilicone-14, acrylates/C12-22 alkyl methacrylate copolymer, acrylates/polytrimethylsiloxymethacrylate copolymer, polyvinylpyrrolidone/vinyl acetate (VP/VA) copolymer, VP/dimethiconylacrylate/polycarbamyl/polyglycol ester, VP/dimethylaminoethylmethacrylate copolymer, VP/dimethylamino ethylmethacrylate/polycarbamyl polyglycol ester, VP/eicosene copolymer, VP/hexadecene copolymer, VP/methacrylamide/vinyl imidazole copolymer, VP/polycarbamyl polyglycol ester, VP/VA copolymer, polyester-1, polyester-2, polyester-3, polyester-4, polyester-5, polyester-7, polyester-8, polyester-10 or a combination thereof. In some embodiments, the film-forming agent is selected from the group consisting of: polymethylsilsesquioxane, acrylates/C12-22 alkyl methacrylate copolymer, and a combination thereof.

In some embodiments, the second film-forming agent is present in an amount of about 1% to about 5% w/w of the ink composition. In some embodiments, second film-forming agent is present in an amount of about 3% w/w of the ink composition. In some embodiments, the second film-forming agent comprises polymethylsilsesquioxane, trimethylsiloxysilicate, polypropylsilsesquioxane, dimethicone, cyclopentasiloxane, dimethiconol crosspolymer, polysilicone-6, polysilicone-8, polysilicone-11, polysilicone-14, acrylates/C12-22 alkyl methacrylate copolymer, acrylates/polytrimethylsiloxymethacrylate copolymer, polyvinylpyrrolidone/vinyl acetate (VP/VA) copolymer, VP/dimethiconylacrylate/polycarbamyl/polyglycol ester, VP/dimethylaminoethylmethacrylate copolymer, VP/dimethylamino ethylmethacrylate/polycarbamyl polyglycol ester, VP/eicosene copolymer, VP/hexadecene copolymer, VP/methacrylamide/vinyl imidazole copolymer, VP/polycarbamyl polyglycol ester, VP/VA copolymer, polyester-1, polyester-2, polyester-3, polyester-4, polyester-5, polyester-7, polyester-8, polyester-10 or a combination thereof. In some embodiments, the second film-forming agent is acrylates/C12-22 alkyl methacrylate copolymer.

In some embodiments, the preservative is present in an amount of about 0.1% to about 5% w/w of the ink composition. In some embodiments, the preservative is present in an amount of about 2% to about 4% w/w of the ink composition. In some embodiments, the preservative is present in an amount of about 3% w/w of the ink composition. In some embodiments, the preservative comprises ascorbic acid, an ascorbate, a palmitate, citric acid, a benzoate, a benzoic acid, a propionate, propionic acid, a sorbate, sorbic acid, a salicylic acid, a salicylate, hexa-2,4-dienoic acid, a hexa-2,4-dienoate, formaldehyde, a formaldehyde releaser, formic acid and its salts, 3-acetyl-6-methylpyran-2,4-(3H)-dione and its salts, 3,3′-dibromo-4,4′- hexamethylenedioxydibenzamidine and its salts, thiomersal, phenylmercuric salts, undec-10-enoic acid and its salts, 1,3-bis (2-ethylhexyl) hexahydro-5-methyl-5-pyrimidine, 5-bromo-5-nitro-1,3-dioxane, bronopol, 2,4-dichlorobenzyl alcohol, 1-(4-chlorophenyl)-3-(3,4-dichlorophenyl) urea, chlorocresol, chloroxylenol, 5-chloro-2-(2,4- dichlorophenoxy) phenol, N,N″-methylenebis[N′-[3-(hydroxymethyl)-2,5- dioxoimidazolidin-4-yl]urea], polyaminopropyl biguanide, methenamine, quaternium-15, climbazole, DMDM hydantoin, benzyl alcohol, 1-Hydroxy-4-methyl-6-(2,4,4-trimethylpentyl)-2-pyridon, piroctone olamine, bromochlorophene, o-cymen-5-ol, chlorophene, chloroacetaminde, methylchloroisothiazolinone, methylisothiazolinone, phenoxyisopropanol, chlorhexidine, chlorhexidine diacetate, chlorhexidine digluconate, chlorhexidine dihydrochloride, dimethyl oxazolidine, behentrimonium chloride, cetri-monium bromide, cetrimonium chloride, laurtrimonium bromide, laurtrimonium chloride, steartrimonium bromide, steartrimonium chloride, diazolidinyl urea, hexamidine, hexamidine diisethionate, hexamidine paraben, glutaral, 7-ethylbicyclooxazolidine, chlorphenesin, sodium hydroxymethylglycinate, silver chloride, benzethonium chloride, benzalkonium chloride, benzalkonium bromide, benzalkonium saccharinate, benzylhemiformal, iodopropynyl butylcarbamate, biphenyl-2-ol and its salts, pyrithionine zinc, an erythorbate, a nitrite, ethylenediaminetetraacetic acid (EDTA), butylated hydroxyanisole (BHA), butylated hydroxytoluene (BHT), capryllic acid, dilauryl thiodipropionate, erythorbic acid, gum guaiac, methylparaben, a sulfite, a bisulfite, a metabisulfite, propyl gallatepy, propylparaben, stannous chloride, sulfur dioxide, thiodipropionic acid, an isothiazoline, a paraben, phenoxyethanol, ethylhexylglycerin, a glycol, a tocopherol, 1,2-hexanediol, or a combination thereof. In some embodiments, the preservative comprises a combination of phenoxyethanol and ethylhexylglycerin.

In some embodiments, the phenoxyethanol and ethylhexylglycerin are present in the composition at a ratio of about 9:1 phenoxyethanol:ethylhexylglycerin. In some embodiments, the combination of phenoxyethanol and ethylhexylglycerin is present in an amount of about 0.1% to about 2% w/w of the ink composition. In some embodiments, the combination of phenoxyethanol and ethylhexylglycerin is present in an amount of about 1% w/w of the ink composition.

In some embodiments, the preservative comprises 1,2-hexanediol. In some embodiments, the 1,2-hexanediol is present in an amount of about 1% to about 3% w/w of the ink composition. In some embodiments, the 1,2-hexanediol is present in an amount of about 2% w/w of the ink composition.

Also provided herein are ink compositions prepared by any of the methods described herein.

As used herein, a “temporary colorant” refers to a colorant that sits on top of the skin or, if it penetrates the skin, can diffuse out of the skin or can be washed off by, for example, water, soap, and/or isopropanol.

As used herein, “color” refers to wavelengths of electromagnetic radiation visible to the human eye.

As used herein, a “colorant” refers to a substance that changes the color of reflected or transmitted light as the result of wavelength-selective absorption.

Reference to the term “about” has its usual meaning in the context of compositions to allow for reasonable variations in amounts that can achieve the same effect and also refers herein to a value of plus or minus 10% of the provided value. For example, “about 20” means or includes amounts from 18 to and including 22.

The details of one or more embodiments of the invention are set forth in the description below. Other features, objects, and advantages of the invention will be apparent from the description and from the claims.

### DETAILED DESCRIPTION

This document provides ink compositions such as genipin-based ink compositions that can be applied to skin as a semi-permanent tattoo. Accordingly, the ink compositions are formulated such that the ink is safe for skin contact and sustains stability or skin transferability for the duration of the product shelf life. Semi-permanent tattoo longevity, or the semi-permanent colorant residence time in the skin, may be limited by the natural skin desquamation process, and the thickness of each layer of the skin (the thickness of the epidermis can depend on the body region). In some embodiments, the ink compositions described herein can be suitable for application to skin using a pen-like applicator.

In some embodiments, the ink compositions provided herein include a semi-permanent colorant; a solvent; and a thickening agent. In some embodiments, the ink compositions provided herein include a semi-permanent colorant; a solvent; and a humectant. In some embodiments, the ink compositions provided herein include a semi-permanent colorant; a solvent; a thickening agent, and a humectant. In some embodiments, the composition further includes a film-forming agent. In some embodiments, the ink compositions provided herein include a semi-permanent colorant; a solvent; and a film-forming agent.

A “semi-permanent colorant” as used herein refers to a colorant that penetrates one or more layers of the skin and is unable to be removed from the skin without physical disruption or natural desquamation of the skin. For example, a semi-permanent colorant may penetrate the stratum corneum and react with other molecules, e.g., other colorant molecules and/or other molecules present in the stratum corneum, such that the semi-permanent colorant is immobilized within the stratum corneum. For example, the semi-permanent colorant may cross-link with collagen found in the stratum corneum. In some embodiments, the semi-permanent colorant may react with other molecules, e.g., other colorant molecules and/or other molecules present in the stratum corneum, to form a molecule that is too large to diffuse out of the stratum corneum. In some embodiments, wherein the semi-permanent colorant penetrates the stratum corneum, the colorant residence time is determined by the natural skin desquamation process. In some embodiments, the semi-permanent colorant cannot be washed off, for example, by water, soap, and/or isopropanol. In some embodiments, a semi-permanent colorant includes a natural or synthetic pigment or dye. In some embodiments, a semi-permanent colorant can include a colorant precursor, for example, a molecule, such as genipin, that expresses color upon reaction with one or more other molecules.

In some embodiments, the semi-permanent colorant (e.g., genipin) is present in an amount of about 0.1% to about 25% w/w of the composition. For example, about 0.1% to about 24%, about 0.1% to about 22%, about 0.1% to about 20%, about 0.1% to about 18%, about 0.1% to about 16%, about 0.1% to about 14%, about 0.1% to about 12%, about 0.1% to about 10%, about 0.1% to about 8%, about 0.1% to about 6%, about 0.1% to about 4%, about 0.1% to about 2%, about 24% to about 25%, about 22% to about 25%, about 20% to about 25%, about 18% to about 25%, about 16% to about 25%, about 14% to about 25%, about 12% to about 25%, about 10% to about 25%, about 8% to about 25%, about 6% to about 25%, about 4% to about 25%, or about 2% to about 25% w/w of the composition. In some embodiments, the semi-permanent colorant (e.g., genipin) is present in an amount of about 5% to about 15%, about 10% to about 15%, about 10% to about 20%, about 15% to about 20%, about 8% to about 12%, about 10% to about 14%, about 12% to about 16%, about 14% to about 18%, or about 16% to about 20% w/w of the composition. In some embodiments, the semi-permanent colorant (e.g., genipin) is present in an amount of about 12% to about 16% w/w of the composition. In some embodiments, the semi-permanent colorant (e.g., genipin) is present in an amount of about 9% to about 11% w/w of the composition. In some embodiments, the semi-permanent colorant (e.g., genipin) is present in an amount of about 1%, about 2%, about 3%, about 4%, about 4.5%, about 5%, about 5.5%, about 6%, about 6.5%, about 7%, about 7.5%, about 8%, about 8.5%, about 9%, about 9.5%, about 10%, about 10.5%, about 11%, about 11.5%, about 12%, about 12.5%, about 13%, about 13.5%, about 14%, about 15%, about 16%, about 17%, about 18%, about 19%, about 20%, about 21%, about 22%, about 23%, about 24%, or about 25% w/w of the composition. In some embodiments, the semi-permanent colorant (e.g., genipin) is present in an amount of about 14% w/w of the composition. In some embodiments, the semi-permanent colorant (e.g., genipin) is present in an amount of about 10% w/w of the composition.

In some embodiments, the semi-permanent colorant includes a colorant precursor. In some embodiments, the colorant precursor is genipin. Accordingly, in some embodiments, the semi-permanent colorant is genipin. In some embodiments, the semi-permanent colorant is purified genipin.

As used herein, “genipin” refers to genipin extract, partially purified genipin extract, and/or purified genipin, as described further herein. Genipin is derived from an iridoid glycoside called geniposide, which is present in nearly 40 plant species. Additionally, genipin is a precursor to gardenia blue that undergoes a free radical polymerization and/or oligomerization reaction upon exposure to amine groups to form a blue colorant (see, for example, Touyama et al., Chem. Pharm. Bull. 42:668-673, 1994; and Touyama et al., Chem. Pharm. Bull. 42(8): 1571-1578, 1994; both of which are incorporated by reference herein in their entireties). When placed in contact with skin, genipin can react with amines in skin to generate the polymer-based and/or oligomer-based blue color in situ. The penetration profile of genipin can dictate the color concentration profile in the stratum corneum as the polymer-based and/or oligomer-based blue colorant can be too large to penetrate the skin at relevant concentrations. For example, the average molecular weight of the polymer-based and/or oligomer-based blue colorant has been reported to be 8900 ± 600 Daltons, and the polymer-based and/or oligomer-based blue colorant can include 40 to 44 monomer units (see, for example, Touyama et al., Chem. Pharm. Bull. 42:668-673, 1994). In some embodiments, the average molecular weight of the blue colorant, for example, polymerized and/or oligomerized genipin produced from the exposure of genipin present in the compositions described herein to amines, can range from about 500 Daltons to about 15000 Daltons. In some embodiments, the average molecular weight of the polymerized and/or oligomerized genipin can range from about 500 Daltons to about 15000 Daltons. In some embodiments, a plurality of different molecular weights of genipin (e.g., including polymerized and/or oligomerized genipin) can be produced from the exposure of genipin present in the compositions described herein to amines. For example, in some embodiments, the polymerized and/or oligomerized genipin can have an average molecular weight from about 500 Daltons to about 15000 Daltons. In some embodiments, the polymerized and/or oligomerized genipin can have an average molecular weight of from about 500 to about 1000 Daltons, about 500 to about 1500 Daltons, about 1000 to about 2000 Daltons, about 1500 to about 2500 Daltons, about 2000 to about 3500 Daltons, about 3500 to about 5000 Daltons, about 5000 to about 6500 Daltons, about 6000 to about 8300 Daltons, or about 8300 to about 9500 Daltons, and combinations thereof.

The presence of amines, for example, amines that are endogenously present in plant extracts or exogenously introduced, in a genipin solution can give rise to shelf life stability challenges. For example, with time, the reactive precursors can develop into the blue semi-permanent colorant (e.g., polymerized and/or oligomerized genipin) prior to application to the skin limiting the penetration of the semi-permanent colorant to the stratum corneum. Penetration of the genipin into the deeper layers of the stratum corneum can allow for the subsequent development and retention of the polymer-based and/or oligomer-based blue semi-permanent colorant in the skin, which may lead to increased semi-permanent tattoo longevity. The semi-permanent tattoo longevity, or the semi-permanent colorant residence time in the skin, may then be limited by the natural skin desquamation process. In some embodiments, purified genipin has a purity of greater than about 85% w/w of the genipin composition. For example, the purified genipin has a purity of greater than about 86%, about 88%, about 90%, about 92%, about 94%, about 96%, about 98%, about 99%, or about 99.5% w/w of the genipin composition.

Non-limiting examples of solvents include water, methanol, ethanol, isopropanol, and diethylene glycol monoethyl ether. In some embodiments, the solvent is a volatile solvent, e.g., the solvent can evaporate or vaporize at room temperature. Non-limiting examples of volatile solvents include water, methanol, ethanol, and isopropanol. In some embodiments, the solvent is selected to have a low toxicity profile when used as described herein. For example, the solvent can be selected such that the amount of any residual solvent left after an ink composition has dried on an applicator is not a toxic amount for contact with a human skin surface.

In some embodiments, the solvent is present in an amount of about 1% to about 95% w/w of the composition. For example, about 10% to about 95%, about 20% to about 95%, about 30% to about 95%, about 40% to about 95%, about 50% to about 95%, about 60% to about 95%, about 70% to about 95%, about 80% to about 95%, about 1% to about 20%, about 1% to about 30%, about 1% to about 40%, about 1% to about 50%, about 1% to about 60%, about 1% to about 70%, about 1% to about 80%, or about 1% to about 90% w/w of the composition. In some embodiments, the solvent is present in an amount of about 50% to about 75%, about 55% to about 80%, about 60% to about 85%, about 65% to about 90%, about 55% to about 75%, about 60% to about 80%, about 65% to about 85%, about 70% to about 90%, about 75% to about 95%, about 60% to about 70%, about 65% to about 75%, about 70% to about 80%, or about 75% to about 85% w/w of the composition. For example, about 60% to about 65%, about 62% to about 67%, about 65% to about 70%, about 68% to about 72%, about 70% to about 75%, about 72% to about 76%, about 75% to about 80%, about 77% to about 82%, about 80% to about 85%, about 82% to about 87%, about 85% to about 90%, about 87% to about 92%, or about 90% to about 95% w/w of the composition. In some embodiments, the solvent is present in an amount of about 76% to about 79% w/w of the composition. In some embodiments, the solvent is present in an amount of about 77% to about 78% w/w of the composition. In some embodiments, the solvent is present in an amount of about 72% to about 76% w/w of the composition. In some embodiments, the solvent is present in an amount of about 69% to about 71% w/w of the composition. In some embodiments, the solvent is present in an amount of about 70% to about 71% w/w of the composition. In some embodiments, the solvent is present in an amount of about 50%, about 55%, about 60%, about 65%, about 66%, about 67%, about 68%, about 69%, about 70%, about 71%, about 72%, about 73%, about 74%, about 75%, about 76%, about 77%, about 78%, about 79%, about 80%, about 85%, about 90%, or about 95% w/w of the composition. In some embodiments, the solvent is present in an amount of about 77% w/w of the composition. In some embodiments, the solvent is present in an amount of about 74% w/w of the composition. In some embodiments, the solvent is present in an amount of about 71% w/w of the composition.

In some embodiments, the solvent is a combination of two or more solvents, e.g., two or more of water, methanol, ethanol, isopropanol, and diethylene glycol monoethyl ether. In some embodiments, each solvent is independently present in an amount of about 5% to about 80% w/w of the composition. For example, each solvent can be independently present in an amount of about 5% to about 10%, about 5% to about 20%, about 5% to about 30%, about 5% to about 40%, about 5% to about 50%, about 5% to about 60%, about 5% to about 70%, about 70% to about 80%, about 60% to about 80%, about 50% to about 80%, about 40% to about 80%, about 30% to about 80%, about 20% to about 80%, or about 10% to about 80% w/w of the composition. In some embodiments, the solvent is independently present in an amount of about 15% to about 25%, about 20% to about 30%, about 25% to about 35%, about 30% to about 40%, about 35% to about 45%, about 40% to about 50%, about 45% to about 50%, or about 50% to about 60% w/w of the composition. For example, each solvent can be independently present in an amount of about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, or about 55% w/w of the composition.

In some embodiments, the solvent is a combination of water and ethanol. In some embodiments, the water is present in an amount of about 15% to about 40% w/w of the composition and the ethanol is present in an amount of about 30% to about 55% w/w of the composition. For example, the water is present in an amount of about 20% to about 30%, about 25% to about 35%, about 25% to about 30%, or about 28% to about 32% w/w of the composition and the ethanol is present in an amount of about 35% to about 45%, about 40% to about 50%, about 40% to about 55%, about 45% to about 55%, about 45% to about 50%, about 40% to about 45%, about 42% to about 47%, or about 45% to about 50% w/w of the composition. In some embodiments, the water is present in an amount of about 25% to about 30% w/w of the composition and the ethanol is present in an amount of about 40% to about 50% w/w of the composition. In some embodiments, the water is present in an amount of about 28% to about 32% w/w of the composition and the ethanol is present in an amount of about 45% to about 50% w/w of the composition. In some embodiments, the water is present in an amount of about 15% to about 25%, about 18% to about 22%, or about 19% to about 21% w/w of the composition and the ethanol is present in an amount of about 45% to about 55%, about 48% to about 52%, or about 49% to about 51% w/w of the composition. In some embodiments, the water is present in an amount of about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, or about 35%, w/w of the composition and the ethanol is present in an amount of about 40%, about 41%, about 42%, about 43%, about 44%, about 45%, about 46%, about 47%, about 48%, about 49%, about 50%, about 51%, about 52%, about 53%, about 54%, or about 55% w/w of the composition. In some embodiments, the water is present in an amount of about 28% w/w of the composition and the ethanol is present in an amount of about 46% w/w of the composition. In some embodiments, the water is present in an amount of about 30% w/w of the composition and the ethanol is present in an amount of about 48% w/w of the composition. In some embodiments, the water is present in an amount of about 21% w/w of the composition and the ethanol is present in an amount of about 50% w/w of the composition.

As used herein, a “thickening agent” refers to an agent that increases the viscosity of a liquid. In some embodiments, the thickening agent increases the viscosity of the liquid without substantially changing other properties of the liquid. Non-limiting examples of thickening agents include starches, gums (e.g., natural and synthetic gums), cellulosics, and arabinogalactan. Non-limiting examples of starches include aluminum starch octenylsuccinate. Non-limiting examples of gums include xanthan gum, sclerotium gum, tragacanth gum, pectin, gum karaya, gum arabic, agar, guar gum, carrageenan, locust bean gum, alginate, alginin, gelatin, tara gum, gum ghatti, gellan gum, konjac gum, cassia gum, spruce gum, chicle gum, dammar, curdlan gum, and pullulan. Non-limiting examples of cellulosics include hydroxyethylcellulose, hydroxypropyl cellulose, hydroxypropyl methylcellulose, ethylcellulose, soybean hemicellulose, and sodium carboxy methylcellulose.

In some embodiments, the thickening agent is present in an amount of about 0.01% to about 1% w/w of the composition. For example about 0.01% to about 0.05%, about 0.01% to about 0.1%, about 0.01% to about 0.15%, about 0.01% to about 0.2%, about 0.01% to about 0.3%, about 0.01% to about 0.4%, about 0.01% to about 0.5%, about 0.01% to about 0.6%, about 0.01% to about 0.7%, about 0.01% to about 0.8%, about 0.01% to about 0.9%, about 0.9% to about 1%, about 0.8% to about 1%, about 0.7% to about 1%, about 0.6% to about 1%, about 0.5% to about 1%, about 0.4% to about 1%, about 0.3% to about 1%, about 0.2% to about 1%, about 0.15% to about 1%, about 0.1% to about 1%, or about 0.05% to about 1% w/w of the composition. In some embodiments, the thickening agent is present in an amount of about 0.05% to about 0.25%, about 0.05% to about 0.2%, about 0.1% to about 0.2%, or about 0.1% to about 0.15% w/w of the composition. In some embodiments, the thickening agent is present in an amount of about 0.05% to about 0.2% w/w of the composition. In some embodiments, the thickening agent is present in an amount of about 0.01%, about 0.05%, 0.06%, about 0.07%, about 0.08%, about 0.09%, about 0.10%, about 0.11%, about 0.12%, about 0.13%, about 0.14%, about 0.15%, about 0.16%, about 0.17%, about 0.18%, about 0.19%, about 0.2%, about 0.22%, about 0.24%, about 0.26%, about 0.28%, about 0.3%, about 0.4%, about 0.5%, about 0.6%, about 0.8%, about 0.9% or about 1% w/w of the composition. In some embodiments, the thickening agent is present in an amount of about 0.12% w/w of the composition.

In some embodiments, the thickening agent is a combination of two or more thickening agents, e.g., two or more of arabinogalactan, aluminum starch octenylsuccinate, xanthan gum, sclerotium gum, tragacanth gum, pectin, gum karaya, gum arabic, agar, guar gum, carrageenan, locust bean gum, alginate, alginin, gelatin, tara gum, gum ghatti, gellan gum, konjac gum, cassia gum, spruce gum, chicle gum, dammar, curdlan gum, pullulan, hydroxyethylcellulose, hydroxypropyl cellulose, hydroxypropyl methylcellulose, ethylcellulose, soybean hemicellulose, and sodium carboxy methylcellulose. In some embodiments, each thickening agent is independently present in an amount of about 0.01% to about 0.5% w/w of the composition. For example, each thickening agent can be independently present in an amount of about 0.01% to about 0.05%, about 0.01% to about 0.1%, about 0.01% to about 0.15%, about 0.01% to about 0.2%, about 0.01% to about 0.25%, about 0.01% to about 0.3%, about 0.01% to about 0.35% w/w of the composition. about 0.01% to about 0.4%, about 0.01% to about 0.45%, about 0.45% to about 0.5%, about 0.4% to about 0.5%, about 0.35% to about 0.5%, about 0.3% to about 0.5%, about 0.25% to about 0.5%, about 0.2% to about 0.5%, about 0.15% to about 0.5%, about 0.1% to about 0.15%, or about 0.05% to about 0.15% w/w of the composition. In some embodiments, each thickening agent is independently present in an amount of about 0.01% to about 0.04%, about 0.02% to about 0.06%, about 0.03% to about 0.07%, 0.04% to about 0.08%, about 0.06% to about 0.1%, or about 0.08% to about 0.12%, or about 0.1% to about 0.14% w/w of the composition. For example, each thickening agent can be independently present in an amount of about 0.01%, about 0.02%, about 0.03%, about 0.04%, about 0.05%, about 0.06%, about 0.07%, about 0.08%, about 0.09%, about 0.1%, about 0.11%, about 0.12%, about 0.13%, about 0.14%, about 0.15%, about 0.16%, about 0.17%, about 0.18%, about 0.19%, about 0.2%, about 0.22%, about 0.24%, about 0.26%, about 0.28%, about 0.3%, about 0.4%, about 0.5%, about 0.6%, about 0.8%, about 0.9% or about 1% w/w of the composition.

In some embodiments, the thickening agent is a combination of hydroxyethylcellulose and xanthan gum. In some embodiments, the hydroxyethylcellulose is present in an amount of about 0.01% to about 0.5% w/w of the composition and xanthan gum is present in an amount of about 0.01% to about 0.5% w/w of the composition. In some embodiments, the hydroxyethylcellulose is present in an amount of about 0.01% to about 0.04%, about 0.02% to about 0.06%, about 0.03% to about 0.07%, 0.04% to about 0.08%, about 0.06% to about 0.1%, or about 0.08% to about 0.12%, or about 0.1% to about 0.14% w/w of the composition and xanthan gum is present in an amount of about about 0.01% to about 0.04%, about 0.02% to about 0.06%, about 0.03% to about 0.07%, 0.04% to about 0.08%, about 0.06% to about 0.1%, or about 0.08% to about 0.12%, or about 0.1% to about 0.14% w/w of the composition. In some embodiments, the hydroxyethylcellulose is present in an amount of about 0.04% to about 0.08% w/w of the composition and xanthan gum is present in an amount of about 0.04% to about 0.08% w/w of the composition. In some embodiments, the hydroxyethylcellulose is present in an amount of about 0.01%, about 0.02%, about 0.03%, about 0.04%, about 0.05%, about 0.06%, about 0.07%, about 0.08%, about 0.09%, about 0.1%, about 0.11%, about 0.12%, about 0.13%, about 0.14%, about 0.15%, about 0.16%, about 0.17%, about 0.18%, about 0.19%, or about 0.2% w/w of the composition and xanthan gum is present in an amount of about 0.01%, about 0.02%, about 0.03%, about 0.04%, about 0.05%, about 0.06%, about 0.07%, about 0.08%, about 0.09%, about 0.1%, about 0.11%, about 0.12%, about 0.13%, about 0.14%, about 0.15%, about 0.16%, about 0.17%, about 0.18%, about 0.19%, or about 0.2% w/w of the composition. In some embodiments, the hydroxyethylcellulose is present in an amount of about 0.06% w/w of the composition and the xanthan gum is present in an amount of about 0.06% w/w of the composition.

A “humectant” as used herein refers to a substance that attracts water. For example, a humectant may attract water to bring moisture to the skin. Non-limiting examples of humectants include polyhydric alcohols, for example, polyalkylene glycols (e.g., alkylene polyols and their derivatives), alpha hydroxy acids, sugars, Aloe vera gel, lithium chloride, allantoin, urea, and dicyanamide. Non-limiting examples of polyhydric alcohols include glycerol, lactic acid, propylene glycol, propanediol, polyethylene glycol, dipropylene glycol, polypropylene glycol, polyethylene glycol and derivatives thereof, hexylene glycol, 1,3-butylene glycol, 1,2,6-hexanetriol, ethoxylated glycerol, propoxylated glycerol, glyceryl triacetate, sorbitol, hydroxypropyl sorbitol, xylitol, maltitol, hyaluronic acid, Tremella extract, sodium lactate, sodium L-pyroglutamate urea, and pyrrolidone carboxylic acid.

In some embodiments, the humectant is present in an amount of about 0.1% to about 20% w/w of the composition. For example, about 0.1% to about 1%, about 0.1% to about 5%, about 0.1% to about 10%, about 0.1% to about 15%, about 15% to about 20%, about 10% to about 20%, about 5% to about 20%, or about 1% to about 20% w/w of the composition. In some embodiments, the humectant is present in an amount of about 0.5% to about 2%, about 0.5% to about 5%, about 1% to about 10%, about 1% to about 5%, about 2% to about 7%, about 5% to about 10%, about 7% to about 12%, or about 10% to about 15% w/w of the composition. In some embodiments, the humectant is present in an amount of about 0.5% to about 1.5% w/w of the composition. In some embodiments, the humectant is present in an amount of about 2% to about 6% w/w of the composition. In some embodiments, the humectant is present in an amount of about 0.1%, about 0.5%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 11%, about 12%, about 13%, about 14%, about 15%, about 16%, about 17%, about 18%, about 19%, or about 20% w/w of the composition. In some embodiments, the humectant is present in an amount of about 4% w/w of the composition. In some embodiments, the humectant is present in an amount of about 1% w/w of the composition.

In some embodiments, the humectant is propanediol. In some embodiments, the propanediol is present in an amount of about 0.1% to about 20% w/w of the composition. For example, about 0.1% to about 1%, about 0.1% to about 5%, about 0.1% to about 10%, about 0.1% to about 15%, about 15% to about 20%, about 10% to about 20%, about 5% to about 20%, or about 1% to about 20% w/w of the composition. In some embodiments, the propanediol is present in an amount of about 0.5% to about 2%, about 0.5% to about 5%, about 1% to about 10%, about 1% to about 5%, about 2% to about 7%, about 5% to about 10%, about 7% to about 12%, or about 10% to about 15% w/w of the composition. In some embodiments, the propanediol is present in an amount of about 0.5% to about 1.5% w/w of the composition. In some embodiments, the propanediol is present in an amount of about 2% to about 6% w/w of the composition. In some embodiments, the propanediol is present in an amount of about 0.1%, about 0.5%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 11%, about 12%, about 13%, about 14%, about 15%, about 16%, about 17%, about 18%, about 19%, or about 20% w/w of the composition. In some embodiments, the propanediol is present in an amount of about 4% w/w of the composition. In some embodiments, the propanediol is present in an amount of about 1% w/w of the composition.

In some embodiments, the humectant is glycerin. In some embodiments, the glycerin is present in an amount of about 0.1% to about 20% w/w of the composition. For example, about 0.1% to about 1%, about 0.1% to about 5%, about 0.1% to about 10%, about 0.1% to about 15%, about 15% to about 20%, about 10% to about 20%, about 5% to about 20%, or about 1% to about 20% w/w of the composition. In some embodiments, the glycerin is present in an amount of about 1% to about 10%, about 1% to about 5%, about 2% to about 7%, about 5% to about 10%, about 7% to about 12%, or about 10% to about 15% w/w of the composition. In some embodiments, the glycerol is present in an amount of about 2% to about 6% w/w of the composition. In some embodiments, the glycerol is present in an amount of about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 11%, about 12%, about 13%, about 14%, about 15%, about 16%, about 17%, about 18%, about 19%, or about 20% w/w of the composition. In some embodiments, the glycerol is present in an amount of about 4% w/w of the composition.

As used herein, a “film-forming agent” refers to a compound that can produce a continuous film on skin. In some embodiments, a film-forming agent is a compound that produces a continuous film on skin upon drying. Non-limiting examples of film-forming agents include volatile silicone resins, polyvinylpyrrolidone, acrylates, acrylamides, copolymers, and isododecane resins. Non-limiting examples of volatile silicone resins include polymethylsilsesquioxane, trimethylsiloxysilicate, polypropylsilsesquioxane, dimethicone, cyclopentasiloxane, dimethiconol crosspolymer, polysilicone-6, polysilicone-8, polysilicone-11, and polysilicone-14. Non-limiting examples of copolymers include acrylates copolymer, styrene/acrylates copolymer, acrylates/C12-22 alkyl methacrylate copolymer, acrylates/polytrimethylsiloxymethacrylate copolymer, polyvinylpyrrolidone/vinyl acetate (VP/VA) copolymer, VP/dimethiconylacrylate/polycarbamyl/polyglycol ester, VP/dimethylaminoethylmethacrylate copolymer, VP/dimethylamino ethylmethacrylate/polycarbamyl polyglycol ester, VP/eicosene copolymer, VP/hexadecene copolymer, VP/methacrylamide/vinyl imidazole copolymer, VP/polycarbamyl polyglycol ester, VP/VA copolymer, polyester-1, polyester-2, polyester-3, polyester-4, polyester-5, polyester-7, polyester-8, and polyester-10.

In some embodiments, the film-forming agent is present in an amount of about 0.1% to about 20% w/w of the composition. For example, about 0.1% to about 1%, about 0.1% to about 5%, about 0.1% to about 10%, about 0.1% to about 15%, about 15% to about 20%, about 10% to about 20%, about 5% to about 20%, or about 1% to about 20% w/w of the composition. In some embodiments, the film-forming agent is present in an amount of about 1% to about 10%, about 1% to about 5%, about 2% to about 7%, about 5% to about 10%, about 7% to about 12%, or about 10% to about 15% w/w of the composition. In some embodiments, the film-forming agent is present in an amount of about 4% to about 6% w/w of the composition. In some embodiments, the film-forming agent is present in an amount of about 6% to about 9% w/w of the composition. In some embodiments, the film-forming agent is present in an amount of about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 11%, about 12%, about 13%, about 14%, about 15%, about 16%, about 17%, about 18%, about 19%, or about 20% w/w of the composition. In some embodiments, the film-forming agent is present in an amount of about 5% w/w of the composition. In some embodiments, the film-forming agent is present in an amount of about 7.5% w/w of the composition.

In some embodiments, the film-forming agent is a combination of two or more film-forming agents. For example, two or more of polymethylsilsesquioxane, trimethylsiloxysilicate, polypropylsilsesquioxane, dimethicone, cyclopentasiloxane, dimethiconol crosspolymer, polysilicone-6, polysilicone-8, polysilicone-11, polysilicone-14, acrylates copolymer, styrene/acrylates copolymer, polyvinylpyrrolidone, acrylates/C12-22 alkyl methacrylate copolymer, acrylates/polytrimethylsiloxymethacrylate copolymer, polyvinylpyrrolidone/vinyl acetate (VP/VA) copolymer, VP/dimethiconylacrylate/polycarbamyl/polyglycol ester, VP/dimethylaminoethylmethacrylate copolymer, VP/dimethylamino ethylmethacrylate/polycarbamyl polyglycol ester, VP/eicosene copolymer, VP/hexadecene copolymer, VP/methacrylamide/vinyl imidazole copolymer, VP/polycarbamyl polyglycol ester, VP/VA copolymer, polyester-1, polyester-2, polyester-3, polyester-4, polyester-5, polyester-7, polyester-8, and polyester-10. In some embodiments, each film-forming agent is independently present in an amount of about 0.1% to about 15% w/w of the composition. For example, about 0.1% to about 1%, about 0.1% to about 5%, about 0.1% to about 10%, about 10% to about 15%, about 5% to about 15%, or about 1% to about 15% w/w of the composition. In some embodiments, each film-forming agent is present in an amount of about 0.1% to about 2%, about 0.5% to about 2%, about 1% to about 10%, about 1% to about 5%, about 2% to about 7%, about 5% to about 10%, about 7% to about 12%, or about 10% to about 15% w/w of the composition. For example, about 0.1%, about 0.25%, about 0.5%, about 0.75%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 11%, about 12%, about 13%, about 14%, about 15%, about 16%, about 17%, about 18%, about 19%, or about 20% w/w of the composition.

In some embodiments, the film-forming agent is a combination of polymethylsilsesquioxane and acrylates/C12-22 alkyl methacrylate copolymer. In some embodiments, the polymethylsilsesquioxane is present in an amount of about 0.5% to about 10% w/w of the composition and the acrylates/C12-22 alkyl methacrylate copolymer is present in an amount of about 0.5% to about 10% w/w of the composition. For example, the polymethylsilsesquioxane is present in an amount of about 0.05% to about 1%, about 0.05% to about 5%, about 0.05% to about 8%, about 8% to about 10%, about 5% to about 10%, about 1% to about 10%, or about 1% to about 5% w/w of the composition and the acrylates/C12-22 alkyl methacrylate copolymer is present in an amount of about 0.05% to about 1%, about 0.05% to about 5%, about 0.05% to about 8%, about 8% to about 10%, about 5% to about 10%, about 1% to about 10%, or about 1% to about 5% w/w of the composition. In some embodiments, the polymethylsilsesquioxane is present in an amount of about 1% to about 5% w/w of the composition and the acrylates/C12-22 alkyl methacrylate copolymer is present in an amount of about 1% to about 5% w/w of the composition. In some embodiments, the polymethylsilsesquioxane is present in an amount of about 0.5%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, or about 10% w/w of the composition and the acrylates/C12-22 alkyl methacrylate copolymer is present in an amount of about 0.5%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, or about 10%w/w of the composition. In some embodiments, the polymethylsilsesquioxane is present in an amount of about 2% w/w of the composition and the acrylates/C12-22 alkyl methacrylate copolymer is present in an amount of about 3% w/w of the composition.

In some embodiments, the film-forming agent is a combination of acrylates copolymer, polyvinylpyrrolidone, and styrene/acrylates copolymer. In some embodiments, the acrylates copolymer is present in an amount of about 0.1% to about 10% w/w of the composition; the polyvinylpyrrolidone is present in an amount of about 0.1% to about 10% w/w of the composition; and the styrene/acrylates copolymer is present in an amount of about 0.1% to about 10% w/w of the composition. In some embodiments, the acrylates copolymer is present in an amount of about 0.1% to about 1%, about 0.1% to about 5%, about 0.1% to about 8%, about 8% to about 10%, about 5% to about 10%, or about 1% to about 10% w/w of the composition; the polyvinylpyrrolidone is present in an amount of about 0.1% to about 1%, about 0.1% to about 5%, about 0.1% to about 8%, about 8% to about 10%, about 5% to about 10%, or about 1% to about 10% w/w of the composition; and the styrene/acrylates copolymer is present in an amount of about 0.1% to about 1%, about 0.1% to about 5%, about 0.1% to about 8%, about 8% to about 10%, about 5% to about 10%, or about 1% to about 10% w/w of the composition. In some embodiments, the acrylates copolymer is present in an amount of about 0.1% to about 1% w/w of the composition; the polyvinylpyrrolidone is present in an amount of about 0.5% to about 1.5% w/w of the composition; and the styrene/acrylates copolymer is present in an amount of about 5% to about 7% w/w of the composition. In some embodiments, the acrylates copolymer is present in an amount of about 0.1%, about 0.25%, about 0.5%, about 1%, about 1.5%, about 2%, about 2.5%, about 3%, about 3.5%, about 4%, about 4.5%, or about 5% w/w of the composition; the polyvinylpyrrolidone is present in an amount of about 0.25%, about 0.5%, about 1%, about 1.5%, about 2%, about 2.5%, about 3%, about 3.5%, about 4%, about 4.5%, about 5%, about 5.5%, or about 6% w/w of the composition; and the styrene/acrylates copolymer is present in an amount of about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, or about 10% w/w of the composition. In some embodiments, the acrylates copolymer is present in an amount of about 0.25% w/w of the composition; the polyvinylpyrrolidone is present in an amount of about 1% w/w of the composition; and the styrene/acrylates copolymer is present in an amount of about 6% w/w of the composition.

In some embodiments, the ink composition can further include a preservative. “Preservative” as used herein refers to an agent that protects against decay, discoloration, and/or spoilage. Non-limiting examples of preservatives include ascorbic acid, ascorbates, palmitate, citric acid, benzoates, benzoic acids, propionates, propionic acid, sorbates, sorbic acid, salicylic acids, salicylates, hexa-2,4-dienoic acid, hexa-2,4-dienoates, formaldehyde, formaldehyde releasers, formic acid and its salts, 3-acetyl-6-methylpyran-2,4-(3H)-dione and its salts, 3,3′-dibromo-4,4′- hexamethylenedioxydibenzamidine and its salts, thiomersal, phenylmercuric salts, undec-10-enoic acid and its salts, 1,3-bis (2-ethylhexyl) hexahydro-5-methyl-5-pyrimidine, 5-bromo-5-nitro-1,3-dioxane, bronopol, 2,4-dichlorobenzyl alcohol, 1-(4-chlorophenyl)-3-(3,4-dichlorophenyl) urea, chlorocresol, chloroxylenol, 5-chloro-2-(2,4- dichlorophenoxy) phenol, N,N″-methylenebis[N′-[3-(hydroxymethyl)-2,5- dioxoimidazolidin-4-yl]urea], polyaminopropyl biguanide, methenamine, quaternium-15, climbazole, DMDM hydantoin, benzyl alcohol, 1-hydroxy-4-methyl-6-(2,4,4-trimethylpentyl)-2-pyridon, piroctone olamine, bromochlorophene, o-cymen-5-ol, chlorophene, chloroacetaminde, methylchloroisothiazolinone, methylisothiazolinone, phenoxyisopropanol, chlorhexidine, chlorhexidine diacetate, chlorhexidine digluconate, chlorhexidine dihydrochloride, dimethyl oxazolidine, behentrimonium chloride, cetri-monium bromide, cetrimonium chloride, laurtrimonium bromide, laurtrimonium chloride, steartrimonium bromide, steartrimonium chloride, diazolidinyl urea, hexamidine, hexamidine diisethionate, hexamidine paraben, glutaral, 7-ethylbicyclooxazolidine, chlorphenesin, sodium hydroxymethylglycinate, silver chloride, benzethonium chloride, benzalkonium chloride, benza-lkonium bromide, benzalkonium saccharinate, benzylhemiformal, iodopropynyl butylcarbamate, biphenyl-2-ol and its salts, pyrithionine zinc, an erythorbate, nitrites, ethylenediaminetetraacetic acid (EDTA), butylated hydroxyanisole (BHA), butylated hydroxytoluene (BHT), capryllic acid, dilauryl thiodipropionate, erythorbic acid, gum guaiac, methylparaben, sulfites, bisulfites, metabisulfites, propyl gallatepy, propylparaben, stannous chloride, sulfur dioxide, thiodipropionic acid, isothiazolines, parabens, phenoxyethanol, ethylhexylglycerin, glycols, and tocopherols.

In some embodiments, the preservative is present in an amount of about 0.05% to about 10% w/w of the composition. For example, about 0.05% to about 0.1%, about 0.05% to about 0.5%, about 0.05% to about 1%, about 0.05% to about 2%, about 0.05% to about 3%, about 0.05% to about 4%, about 0.05% to about 5%, about 0.05% to about 6%, about 0.05% to about 7%, about 0.05% to about 8%, about 0.05% to about 9%, about 9% to about 10%, about 8% to about 10%, about 7% to about 10%, about 6% to about 10%, about 5% to about 10%, about 4% to about 10%, about 3% to about 10%, about 2% to about 10%, about 1% to about 10%, about 0.5% to about 10%, or about 0.1% to about 10% w/w of the composition. In some embodiments, the preservative is present in an amount of about 1% to about 5% w/w of the composition. In some embodiments, the preservative is present in an amount of 0.1% to about 2%, 0.5% to about 2.5%, about 1% to about 3%, about 1.5% to about 3.5%, about 2% to about 4%, about 2.5% to about 4.5%, or about 3% to about 5% w/w of the composition. In some embodiments, the preservative is present in an amount of about 0.5% to about 1.5%, about 0.5% to about 1.0%, about 1.5% to about 2%, about 1% to about 2%, about 0.5% to about 2%, or about 0.8% to about 2% w/w of the composition. For example, about 0.05% to about 0.4%, about 0.05% to about 0.2%, about 0.2% to about 0.5%, about 0.15% to about 0.25%, or about 0.1% to about 0.3% w/w of the composition. In some embodiments, the preservative is present in an amount of about 0.01%, about 0.05%, about 0.1%, about 0.5%, about 1%, about 1.5%, about 2%, about 2.5%, about 3%, about 3.5%, about 4%, about 4.5%, about 5%, about 5.5%, or about 6% w/w of the composition. In some embodiments, the preservatives is present in an amount of about 3% w/w of the composition. In some embodiments, the preservatives is present in an amount of about 1% w/w of the composition.

In some embodiments, the preservative is a combination of two or more preservatives. For example, two or more of ascorbic acid, an ascorbate, a palmitate, citric acid, a benzoate, a benzoic acid, a propionate, propionic acid, a sorbate, sorbic acid, a salicylic acid, a salicylate, hexa-2,4-dienoic acid, a hexa-2,4-dienoate, formaldehyde, a formaldehyde releaser, formic acid and its salts, 3-acetyl-6-methylpyran-2,4-(3H)-dione and its salts, 3,3′-dibromo-4,4′- hexamethylenedioxydibenzamidine and its salts, thiomersal, phenylmercuric salts, undec-10-enoic acid and its salts, 1,3-bis (2-ethylhexyl) hexahydro-5-methyl-5-pyrimidine, 5-bromo-5-nitro-1,3-dioxane, bronopol, 2,4-dichlorobenzyl alcohol, 1-(4-chlorophenyl)-3-(3,4-dichlorophenyl) urea, chlorocresol, chloroxylenol, 5-chloro-2-(2,4- dichlorophenoxy) phenol, N,N″-methylenebis[N′-[3-(hydroxymethyl)-2,5- dioxoimidazolidin-4-yl]urea], polyaminopropyl biguanide, methenamine, quaternium-15, climbazole, DMDM hydantoin, benzyl alcohol, 1-Hydroxy-4-methyl-6-(2,4,4-trimethylpentyl)-2-pyridon, piroctone olamine, bromochlorophene, o-cymen-5-ol, chlorophene, chloroacetaminde, methylchloroisothiazolinone, methylisothiazolinone, phenoxyisopropanol, chlorhexidine, chlorhexidine diacetate, chlorhexidine digluconate, chlorhexidine dihydrochloride, dimethyl oxazolidine, behentrimonium chloride, cetri-monium bromide, cetrimonium chloride, laurtrimonium bromide, laurtrimonium chloride, steartrimonium bromide, steartrimonium chloride, diazolidinyl urea, hexamidine, hexamidine diisethionate, hexamidine paraben, glutaral, 7-ethylbicyclooxazolidine, chlorphenesin, sodium hydroxymethylglycinate, silver chloride, benzethonium chloride, benzalkonium chloride, benza-lkonium bromide, benzalkonium saccharinate, benzylhemiformal, iodopropynyl butylcarbamate, biphenyl-2-ol and its salts, pyrithionine zinc, an erythorbate, a nitrite, ethylenediaminetetraacetic acid (EDTA), sodium lignosulfonate, butylated hydroxyanisole (BHA), butylated hydroxytoluene (BHT), capryllic acid, dilauryl thiodipropionate, erythorbic acid, gum guaiac, methylparaben, a sulfite, a bisulfite, a metabisulfite, propyl gallatepy, propylparaben, stannous chloride, sulfur dioxide, thiodipropionic acid, an isothiazoline, a paraben, phenoxyethanol, ethylhexylglycerin, a glycols, and a tocopherol. In some embodiments, each preservative is independently present in an amount of about 0.01% to about 8% w/w of the composition. For example, about 0.01% to about 0.05%, about 0.01% to about 0.1%, about 0.01% to about 0.5%, about 0.01% to about 1%, about 0.01% to about 2%, about 0.01% to about 3%, about 0.01% to about 4%, about 0.01% to about 5%, about 0.01% to about 6%, about 0.01% to about 7%, about 7% to about 8%, about 6% to about 8%, about 5% to about 8%, about 4% to about 8%, about 3% to about 8%, about 2% to about 8%, about 1% to about 8%, about 0.5% to about 8%, or about 0.1% to about 8% w/w of the composition. In some embodiments, each preservative is independently present in an amount of about 0.05% to about 8% w/w of the composition. For example, about 0.05% to about 0.1%, about 0.05% to about 0.5%, about 0.05% to about 1%, about 0.05% to about 2%, about 0.05% to about 3%, about 0.05% to about 4%, about 0.05% to about 5%, about 0.05% to about 6%, about 0.05% to about 7%, about 7% to about 8%, about 6% to about 8%, about 5% to about 8%, about 4% to about 8%, about 3% to about 8%, about 2% to about 8%, about 1% to about 8%, about 0.5% to about 8%, or about 0.1% to about 8% w/w of the composition. In some embodiments, the each preservative is independently present in an amount of about 1% to about 5% w/w of the composition. In some embodiments, each preservative is independently present in an amount of about 0.025% to about 0.1%, about 0.1% to about 2%, 0.5% to about 2.5%, about 1% to about 3%, about 1.5% to about 3.5%, about 2% to about 4%, about 2.5% to about 4.5%, or about 3% to about 5% w/w of the composition. In some embodiments, each preservative is independently present in an amount of about 0.5% to about 1.5%, about 0.5% to about 1.0%, about 1.5% to about 2%, about 1% to about 2%, about 0.5% to about 2%, or about 0.8% to about 2% w/w of the composition. In some embodiments, each preservative is independently present in an amount of about 0.01%, about 0.05%, about 0.1%, about 0.5%, about 1%, about 1.5%, about 2%, about 2.5%, about 3%, about 3.5%, about 4%, about 4.5%, about 5%, about 5.5%, or about 6% w/w of the composition.

In some embodiments, the preservative includes a combination of phenoxyethanol and ethylhexylglycerin. In some embodiments, the preservative further includes 1,2-hexanediol (e.g., the preservative is a combination of phenoxyethanol, ethylhexylglycerin, and 1,2-hexanediol). In some embodiments, the preservative further includes EDTA (e.g., the preservative is a combination of phenoxyethanol, ethylhexylglycerin, and EDTA). In some embodiments, the phenoxyethanol is present in an amount of about 0.1% and 5% w/w of the composition; the ethylhexylglycerin is present in an amount of about 0.01% to about 3% w/w of the composition; and the 1,2-hexanediol is present in an amount of about 0.1% to about 5% w/w of the composition. In some embodiments, the phenoxyethanol is present in an amount of about 0.1% to about 0.5%, about 0.1% to about 1%, about 0.1% to about 2%, about 0.1% to about 3%, about 0.1% to about 4%, about 4% to about 5%, about 3% to about 5%, about 2% to about 5%, about 1% to about 5%, about 0.5% to about 5%, about 0.5% to about 1%, about 0.5% to about 2.5%, about 1% to about 3%, about 1.5% to about 3.5%, about 2% to about 4%, or about 2.5% to about 4.5% w/w of the composition; the ethylhexylglycerin is present in an amount of about 0.01% to about 0.1%, 0.01% to about 0.5%, 0.01% to about 1%, about 0.01% to about 2%, about 2% to about 3%, about 1% to about 3%, about 0.1% to about 3%, about 0.05% to about 3%, about 0.05% to about 0.15%, about 0.05% to about 1%, 0.5% to about 2.5%, about 1% to about 3%, about 1.5% to about 3.5%, about 2% to about 4%, about 2.5% to about 4.5%, or about 3% to about 5% w/w of the composition; and the 1,2-hexanediol is present in an amount of about 0.1% to about 1%, about 0.1% to about 2%, about 0.1% to about 3%, about 0.1% to about 4%, about 4% to about 5%, about 3% to about 5%, about 2% to about 5%, about 1% to about 5%, 0.5% to about 2.5%, about 1% to about 3%, about 1.5% to about 3.5%, about 2% to about 4%, about 2.5% to about 4.5%, or about 3% to about 5%w/w of the composition. In some embodiments, the phenoxyethanol is present in an amount of about 0.5%, about 0.6%, about 0.7%, about 0.8%, about 0.9%, about 1%, about 1.1%, about 1.2%, about 1.3%, about 1.4%, about 1.5%, about 2%, about 2.5%, about 3%, about 3.5%, about 4%, or about 4.5% w/w of the composition; the ethylhexylglycerin is present in an amount of about 0.01%, about 0.05%, about 0.06%, about 0.07%, about 0.08%, about 0.09%, about 0.1%, about 0.12%, about 0.13%, about 0.14%, about 0.15%, about 0.25%, about 0.5%, about 1%, about 1.5%, about 2%, or about 2.5% w/w of the composition; and the 1,2-hexanediol is present in an amount of about 0.5%, about 1%, about 1.5%, about 2%, about 2.5%, about 3%, about 3.5%, about 4%, about 4.5%, about 5%, about 5.5%, or about 6% w/w of the composition.

In some embodiments, the preservative further includes EDTA (e.g., the preservative is a combination of phenoxyethanol, ethylhexylglycerin, and EDTA). In some embodiments, the phenoxyethanol is present in an amount of about 0.1% and 5% w/w of the composition; the ethylhexylglycerin is present in an amount of about 0.01% to about 3% w/w of the composition; and the EDTA is present in an amount of about 0.01% to about 2% w/w of the composition. In some embodiments, the phenoxyethanol is present in an amount of about 0.1% to about 0.5%, about 0.1% to about 1%, about 0.1% to about 2%, about 0.1% to about 3%, about 0.1% to about 4%, about 4% to about 5%, about 3% to about 5%, about 2% to about 5%, about 1% to about 5%, about 0.5% to about 5%, about 0.5% to about 1%, about 0.5% to about 2.5%, about 1% to about 3%, about 1.5% to about 3.5%, about 2% to about 4%, or about 2.5% to about 4.5% w/w of the composition; the ethylhexylglycerin is present in an amount of about 0.01% to about 0.1%, 0.01% to about 0.5%, 0.01% to about 1%, about 0.01% to about 2%, about 2% to about 3%, about 1% to about 3%, about 0.1% to about 3%, about 0.05% to about 3%, about 0.05% to about 0.15%, about 0.05% to about 1%, 0.5% to about 2.5%, about 1% to about 3%, about 1.5% to about 3.5%, about 2% to about 4%, about 2.5% to about 4.5%, or about 3% to about 5% w/w of the composition; and the EDTA is present in an amount of about 0.01% to about 0.05%, about 0.01% to about 0.1%, about 0.01% to about 0.5%, about 0.01% to about 1%, about 0.1% to about 1.5%, about 1.5% to about 2%, about 1% to about 2%, about 0.5% to about 2%, about 0.1% to about 2%, or about 0.05% to about 2% w/w of the composition. In some embodiments, the phenoxyethanol is present in an amount of about 0.5%, about 0.6%, about 0.7%, about 0.8%, about 0.9%, about 1%, about 1.1%, about 1.2%, about 1.3%, about 1.4%, about 1.5%, about 2%, about 2.5%, about 3%, about 3.5%, about 4%, or about 4.5% w/w of the composition; the ethylhexylglycerin is present in an amount of about 0.01%, about 0.05%, about 0.06%, about 0.07%, about 0.08%, about 0.09%, about 0.1%, about 0.12%, about 0.13%, about 0.14%, about 0.15%, about 0.25%, about 0.5%, about 1%, about 1.5%, about 2%, or about 2.5% w/w of the composition; and the EDTA is present in an amount of about 0.01%, about 0.05%, about 0.1%, about 0.5%, about 1%, about 1.5%, or about 2% w/w of the composition.

In some embodiments, the phenoxyethanol and ethylhexylglycerin are present in the composition at a ratio of about 9:1 phenoxyethanol:ethylhexylglycerin (e.g., EUXYL ® PE9010).

In some embodiments, the EDTA is disodium EDTA.

In some embodiments, the preservative further includes sodium lignosulfonate.

In some embodiments, the ink composition can further include one or more temporary colorants. In some embodiments, the one or more temporary colorants are present in an amount of about 0.1% to about 10% w/w of the composition. For example, about 0.1% to about 1%, about 0.1% to about 5%, about 0.1% to about 8%, about 8% to about 10%, about 5% to about 10%, or about 1% to about 10% w/w of the composition. In some embodiments, the one or more temporary colorant are present in an amount of about 2% to about 3% w/w of the composition. In some embodiments, the one or more temporary colorant are present in an amount of about 2.5% w/w of the composition. In some embodiments, the one or more temporary colorants are selected from the group consisting of: iron oxide black (Fe₃O4), iron oxide (FeO), carbon, logwood, ochre, cinnabar (HgS), cadmium red (CdSe), iron (III) oxide (Fe₂O₃), naphthol-AS pigment, disazodiarylide, disazopyrazolone, cadmium seleno-sulfide, cadmium yellow (CdS, CdZnS), curcuma yellow, chrome yellow (PbCrO4), disazodiarylide, chromium oxide (Cr₂O₃), malachite [Cu₂(CO₃)(OH)₂], a ferrocyanide, a ferricyanide, lead chromate, monoazo pigment, Cu/Al phthalocyanine, Cu phthalocyanine, azure blue, cobalt blue, Cu-phthalocyanine, manganese violet (manganese ammonium pyrophosphate), an aluminum salt, quinacridone, dioxazine/carbazole, lead white (lead carbonate), titanium dioxide (TiO₂), barium sulfate (BaSO₄), zinc oxide, anthraquinone dyes and derivatives, annatto, caramel, β-carotene, bismuth citrate, disodium EDTA copper, potassium sodium copper chlorophyllin, dihydroxyacetone, bismuth oxychloride, guaiazulene, henna, ferric ammonium ferrocyanide, ferric ferrocyanide, chromium hydroxide green, chromium oxide greens, guanine, lead acetate, pyrophillite, mica, silver, titanium dioxide, aluminum powder, bronze powder, copper powder, ultramarines, manganese violet, zinc oxide, luminescent zinc sulfide, D&C Black Nos. 2 and 3, FD&C Blue No. 1 (e.g., acid blue 9), D&C Blue No. 4, D&C Brown No. 1, FD&C Green No. 3, D&C Green Nos. 5, 6, and 8, D&C Orange Nos. 4, 5, 10 and 11, FD&C Red Nos. 4, D&C Red Nos. 6, 7, 17, 21, 22, 27, 28, 30, 32, 33, 34, 36 and 40, D&C Violet No. 2, Ext. D&C Violet No.2, FD&C Yellow Nos. 5 and 6, D&C Yellow Nos. 7, 8, 10 and 11, and Ext. D&C Yellow No. 7. In some embodiments, the temporary colorant does not contain an amine group. In some embodiments, the temporary colorant is D&C Black No. 2.

In some embodiments, a composition as provided herein includes:
genipin present in an amount of about 10% to about 20% w/w of the composition;
a solvent present in an amount of about 65% to about 80% w/w of the composition; and
a thickening agent present in an amount of about 0.01% to about 0.3% w/w of the composition. In some embodiments, the solvent is a combination of water and ethanol.
In some such embodiments, the water is present in an amount of about 25% to about 30% w/w of the composition, and the ethanol is present in an amount of about 40% to about 50% w/w of the composition. In some embodiments, the thickening agent is a combination of hydroxyethylcellulose and xanthan gum. In some such embodiments, the hydroxyethylcellulose is present in an amount of about 0.01% to about 0.1% w/w of the composition, and the xanthan gum is present in an amount of about 0.01% to about 0.1% w/w of the composition.

In some embodiments, the composition further includes a humectant present in an amount of about 1% to about 10% w/w of the composition. In some embodiments, the humectant is glycerol.

In some embodiments, the composition further includes a film-forming agent present in an amount of about 0.1% to about 10% w/w of the composition. In some embodiments, the film-forming agent is a combination of polymethylsilsesquioxane and acrylates/C12-22 alkyl methacrylate copolymer. In some such embodiments, the polymethylsilsesquioxane is present in an amount of about 1% to about 5% w/w of the composition, and the acrylates/C12-22 alkyl methacrylate copolymer is present in an amount of about 1% to about 5% w/w of the composition.

In some embodiments, the composition further includes a preservative present in an amount of about 0.1% to about 5% w/w of the composition. In some embodiments, the preservative includes a combination of phenoxyethanol and ethylhexylglycerin. In some embodiments, the preservative includes a combination of phenoxyethanol, ethylhexylglycerin, and 1,2-hexanediol. In some such embodiments, the phenoxyethanol and ethylhexylglycerin are together present in the composition in an amount of about 0.1% to about 2% w/w of the composition. In some such embodiments, the phenoxyethanol and ethylhexylglycerin are present in the composition at a ratio of about 9:1 phenoxyethanol:ethylhexylglycerin. In some such embodiments, the 1,2-hexanediol is present in an amount of about 1% to about 3% w/w of the composition.

In some embodiments, a composition as provided herein includes:
genipin present in an amount of about 10% to about 20% w/w of the composition;
a solvent present in an amount of about 65% to about 80% w/w of the composition; and
a humectant present in an amount of about 1% to about 10% w/w of the composition. In some embodiments, the solvent is a combination of water and ethanol. In some embodiments, the water is present in an amount of about 25% to about 30% w/w of the composition. In some embodiments, the ethanol is present in an amount of about 40% to about 50% w/w of the composition. In some embodiments, the humectant is glycerol.

In some embodiments, the composition further includes a thickening agent present in an amount of about 0.01% to about 0.3% w/w of the composition. In some embodiments, the thickening agent is a combination of hydroxyethylcellulose and xanthan gum. In some such embodiments, the hydroxyethylcellulose is present in an amount of about 0.01% to about 0.1% w/w of the composition, and the xanthan gum is present in an amount of about 0.01% to about 0.1% w/w of the composition.

In some embodiments, the composition further includes a film-forming agent present in an amount of about 0.1% to about 10% w/w of the composition. In some embodiments, the film-forming agent is a combination of polymethylsilsesquioxane and acrylates/C12-22 alkyl methacrylate copolymer. In some such embodiments, the polymethylsilsesquioxane is present in an amount of about 1% to about 5% w/w of the composition, and the acrylates/C12-22 alkyl methacrylate copolymer is present in an amount of about 1% to about 5% w/w of the composition. In some embodiments, the composition further includes a humectant present in an amount of about 1% to about 10% w/w of the composition. In some embodiments, the humectant is glycerol.

In some embodiments, the composition further includes a preservative present in an amount of about 0.1% to about 5% w/w of the composition. In some embodiments, the preservative includes a combination of phenoxyethanol and ethylhexylglycerin. In some embodiments, the preservative includes a combination of phenoxyethanol, ethylhexylglycerin, and 1,2-hexanediol. In some such embodiments, the phenoxyethanol and ethylhexylglycerin are together present in the composition in an amount of about 0.1% to about 2% w/w of the composition. In some such embodiments, the phenoxyethanol and ethylhexylglycerin are present in the composition at a ratio of about 9:1 phenoxyethanol:ethylhexylglycerin. In some such embodiments, the 1,2-hexanediol is present in an amount of about 1% to about 3% w/w of the composition.

In some embodiments, a composition as provided herein includes:
genipin present in an amount of about 12% to about 16% w/w of the composition;
a solvent present in an amount of about 72% to about 76% w/w of the composition; and
a thickening agent present in an amount of about 0.05% to about 0.20% w/w of the composition. In some embodiments, the solvent is a combination of water and ethanol.
In some such embodiments, the water is present in an amount of about 25% to about 30% w/w of the composition, and the ethanol is present in an amount of about 45% to about 47% w/w of the composition. In some embodiments, the thickening agent is a combination of hydroxyethylcellulose and xanthan gum. In some such embodiments, the hydroxyethylcellulose is present in an amount of about 0.04% to about 0.08% w/w of the composition, and the xanthan gum is present in an amount of about 0.04% to about 0.08% w/w of the composition.

In some embodiments, the composition further includes a film-forming agent present in an amount of about 4% to about 6% w/w of the composition. In some embodiments, the film-forming agent is a combination of polymethylsilsesquioxane and acrylates/C12-22 alkyl methacrylate copolymer. In some such embodiments, the polymethylsilsesquioxane is present in an amount of about 1% to about 5% w/w of the composition, and the acrylates/C12-22 alkyl methacrylate copolymer is present in an amount of about 1% to about 5% w/w of the composition.

In some embodiments, the composition further includes a humectant present in an amount of about 2% to about 6% w/w of the composition. In some embodiments, the humectant is glycerol.

In some embodiments, the composition further includes a preservative present in an amount of 2% to about 4% w/w of the composition. In some embodiments, the preservative includes a combination of phenoxyethanol and ethylhexylglycerin. In some embodiments, the preservative includes a combination of phenoxyethanol, ethylhexylglycerin, and 1,2-hexanediol. In some such embodiments, the phenoxyethanol and ethylhexylglycerin are together present in the composition in an amount of about 0.1% to about 2% w/w of the composition. In some such embodiments, the phenoxyethanol and ethylhexylglycerin are present in the composition at a ratio of about 9:1 phenoxyethanol:ethylhexylglycerin. In some such embodiments, the 1,2-hexanediol is present in an amount of about 1% to about 3% w/w of the composition.

In some embodiments, a composition as provided herein includes:
genipin present in an amount of about 12% to about 16% w/w of the composition;
a solvent present in an amount of about 72% to about 76% w/w of the composition; and
a humectant present in an amount of about 2% to about 6% w/w of the composition. In some embodiments, the solvent is a combination of water and ethanol. In some such embodiments, the water is present in an amount of about 25% to about 30% w/w of the composition, and the ethanol is present in an amount of about 45% to about 47% w/w of the composition. In some embodiments, the humectant is glycerol.

In some embodiments, the composition further includes a thickening agent present in an amount of about 0.05% to about 0.20% w/w of the composition. In some embodiments, the thickening agent is a combination of hydroxyethylcellulose and xanthan gum. In some such embodiments, the hydroxyethylcellulose is present in an amount of about 0.04% to about 0.08% w/w of the composition, and the xanthan gum is present in an amount of about 0.04% to about 0.08% w/w of the composition.

In some embodiments, the composition further includes a film-forming agent present in an amount of about 4% to about 6% w/w of the composition. In some embodiments, the film-forming agent is a combination of polymethylsilsesquioxane and acrylates/C12-22 alkyl methacrylate copolymer. In some such embodiments, the polymethylsilsesquioxane is present in an amount of about 1% to about 5% w/w of the composition, and the acrylates/C12-22 alkyl methacrylate copolymer is present in an amount of about 1% to about 5% w/w of the composition.

In some embodiments, the composition further includes a preservative present in an amount of 2% to about 4% w/w of the composition. In some embodiments, the preservative includes a combination of phenoxyethanol and ethylhexylglycerin. In some embodiments, the preservative includes a combination of phenoxyethanol, ethylhexylglycerin, and 1,2-hexanediol. In some such embodiments, the phenoxyethanol and ethylhexylglycerin are together present in the composition in an amount of about 0.1% to about 2% w/w of the composition. In some such embodiments, the phenoxyethanol and ethylhexylglycerin are present in the composition at a ratio of about 9:1 phenoxyethanol:ethylhexylglycerin. In some such embodiments, the 1,2-hexanediol is present in an amount of about 1% to about 3% w/w of the composition.

In some embodiments, a composition as provided herein includes:
genipin present in an amount of about 14% w/w of the composition;
a solvent present in an amount of about 74% w/w of the composition; and
a thickening agent present in an amount of about 0.12% w/w of the composition.
In some embodiments, the solvent is a combination of water and ethanol. In some such embodiments, the water is present in an amount of about 28% w/w of the composition, and the ethanol is present in an amount of about 46% w/w of the composition. In some embodiments, the thickening agent is a combination of hydroxyethylcellulose and xanthan gum. In some embodiments, the hydroxyethylcellulose is present in an amount of about 0.06% w/w of the composition, and the xanthan gum is present in an amount of about 0.06% w/w of the composition.

In some embodiments, the composition further includes a film-forming agent present in an amount of about 5% w/w of the composition. In some embodiments, the film-forming agent is a combination of polymethylsilsesquioxane and acrylates/C12-22 alkyl methacrylate copolymer. In some such embodiments, the polymethylsilsesquioxane is present in an amount of about 2% w/w of the composition, and the acrylates/C12-22 alkyl methacrylate copolymer is present in an amount of about 3% w/w of the composition. In some embodiments, the composition further includes a humectant present in an amount of about 4% w/w of the composition. In some embodiments, the humectant is glycerol.

In some embodiments, the composition further includes a preservative present in an amount of 3% w/w of the composition. In some embodiments, the preservative includes a combination of phenoxyethanol and ethylhexylglycerin. In some embodiments, the preservative includes a combination of phenoxyethanol, ethylhexylglycerin, and 1,2-hexanediol. In some such embodiments, the phenoxyethanol and ethylhexylglycerin are together present in the composition in an amount of about 1% w/w of the composition. In some such embodiments, the phenoxyethanol and ethylhexylglycerin are present in the composition at a ratio of about 9:1 phenoxyethanol:ethylhexylglycerin. In some such embodiments, the 1,2-hexanediol is present in an amount of about 2% w/w of the composition.

In some embodiments, a composition as provided herein includes:
genipin present in an amount of about 14% w/w of the composition;
a solvent present in an amount of about 74% w/w of the composition; and
a humectant present in an amount of about 4% w/w of the composition. In some embodiments, the solvent is a combination of water and ethanol. In some such embodiments, the water is present in an amount of about 28% w/w of the composition, and the ethanol is present in an amount of about 46% w/w of the composition. In some embodiments, the humectant is glycerol.

In some embodiments, the composition further includes a thickening agent present in an amount of about 0.12% w/w of the composition. In some embodiments, the thickening agent is a combination of hydroxyethylcellulose and xanthan gum. In some such embodiments, the hydroxyethylcellulose is present in an amount of about 0.06% w/w of the composition, and the xanthan gum is present in an amount of about 0.06% w/w of the composition.

In some embodiments, the composition further includes a film-forming agent present in an amount of about 5% w/w of the composition. In some embodiments, the film-forming agent is a combination of polymethylsilsesquioxane and acrylates/C12-22 alkyl methacrylate copolymer. In some such embodiments, the polymethylsilsesquioxane is present in an amount of about 2% w/w of the composition, and the acrylates/C12-22 alkyl methacrylate copolymer is present in an amount of about 3% w/w of the composition.

In some embodiments, the composition further includes a preservative present in an amount of 3% w/w of the composition. In some embodiments, the preservative includes a combination of phenoxyethanol and ethylhexylglycerin. In some embodiments, the preservative includes a combination of phenoxyethanol, ethylhexylglycerin, and 1,2-hexanediol. In some such embodiments, the phenoxyethanol and ethylhexylglycerin are together present in the composition in an amount of about 1% w/w of the composition. In some such embodiments, the phenoxyethanol and ethylhexylglycerin are present in the composition at a ratio of about 9:1 phenoxyethanol:ethylhexylglycerin. In some such embodiments, the 1,2-hexanediol is present in an amount of about 2% w/w of the composition.

In some embodiments, a composition as provided herein includes:
genipin present in an amount of about 5% to about 15% w/w of the composition;
a solvent present in an amount of about 65% to about 85% w/w of the composition; and
a film-forming agent present in an amount of about 5% to about 10% w/w of the composition.

In some embodiments, a composition as provided herein includes:
genipin present in an amount of about 9% to about 11% w/w of the composition;
a solvent present in an amount of about 75% to about 80% w/w of the composition; and
a film-forming agent present in an amount of about 6% to about 9% w/w of the composition.

In some embodiments, the solvent is a combination of water and ethanol. In some such embodiments, the water is present in an amount of about 25% to about 35% w/w of the composition, and the ethanol is present in an amount of about 45% to about 50% w/w of the composition. In some embodiments, the film-forming agent is a combination of acrylates copolymer, polyvinylpyrrolidone, and styrene/acrylates copolymer.

In some embodiments, the composition further includes a humectant present in an amount of about 0.5% to about 1.5% w/w of the composition. In some embodiments, the humectant is propanediol.

In some embodiments, the composition further includes a preservative present in an amount of 0.5% to about 1.5% w/w of the composition. In some embodiments, the preservative includes a combination of phenoxyethanol and ethylhexylglycerin. In some embodiments, the preservative includes a combination of phenoxyethanol, ethylhexylglycerin, and EDTA. In some such embodiments, the phenoxyethanol and ethylhexylglycerin are together present in the composition in an amount of about 0.1% to about 2% w/w of the composition. In some such embodiments, the phenoxyethanol and ethylhexylglycerin are present in the composition at a ratio of about 9:1 phenoxyethanol:ethylhexylglycerin. In some such embodiments, the EDTA is present in an amount of about 0.05% w/w of the composition.

In some embodiments, a composition as provided herein includes:
genipin present in an amount of about 10% w/w of the composition;
a solvent present in an amount of about 77% w/w of the composition; and
a film-forming agent present in an amount of about 7.5% w/w of the composition.

In some embodiments, the solvent is a combination of water and ethanol. In some such embodiments, the water is present in an amount of about 30% w/w of the composition, and the ethanol is present in an amount of about 48% w/w of the composition. In some embodiments, the film-forming agent is a combination of acrylates copolymer, polyvinylpyrrolidone, and styrene/acrylates copolymer.

In some embodiments, the composition further includes a humectant present in an amount of about 1% w/w of the composition. In some embodiments, the humectant is propanediol.

In some embodiments, the composition further includes a preservative present in an amount of about 1.05% w/w of the composition. In some embodiments, the preservative includes a combination of phenoxyethanol and ethylhexylglycerin. In some embodiments, the preservative includes a combination of phenoxyethanol, ethylhexylglycerin, and EDTA. In some such embodiments, the phenoxyethanol and ethylhexylglycerin are together present in the composition in an amount of about 1% w/w of the composition. In some such embodiments, the phenoxyethanol and ethylhexylglycerin are present in the composition at a ratio of about 9:1 phenoxyethanol:ethylhexylglycerin. In some such embodiments, the EDTA is present in an amount of about 0.05% w/w of the composition.

Also provided herein are methods of manufacturing an ink composition, e.g., any of the ink compositions described herein. For example, such methods can include i) combining a thickening agent with a first solvent to form a first solution; ii) combining genipin with a second solvent to form a second solution; and iii) combining the first solution and second solution to form an ink composition. In some embodiments, methods of manufacturing an ink composition as described herein include: i) combining a thickening agent and a humectant with a first solvent to form a first solution; ii) combining genipin and a film-forming agent (e.g., a first film-forming agent) with a second solvent to form a second solution; and iii) combining the first solution and second solution to form an ink composition. In some embodiments, the methods described herein further include: iv) adding a film-forming agent (e.g., a first or second film-forming agent) agent and/or a preservative to the ink composition.

In some embodiments, the first solvent is any of the solvents described herein. In some embodiments, the second solvent is any of the solvents described herein. In some embodiments, the first solvent and second solvent are different.

In some embodiments, the first solvent is water. In some embodiments, the second solvent is ethanol.

In some embodiments, the thickening agent (e.g., any of the thickening agents described herein) and first solvent are present in a ratio of about 1:500 to about 4:500 thickening agent:first solvent in the first solution. In some embodiments, the thickening agent and first solvent are present in a ratio of about 2:500 thickening agent:first solvent in the first solution.

In some embodiments, the thickening agent is a combination of hydroxyethylcellulose and xanthan gum. In some embodiments, the combination of hydroxyethylcellulose and xanthan gum is present at a ratio of about 2:1 to about 1:2 hydroxyethylcellulose:xanthan gum. In some embodiments, the combination of hydroxyethylcellulose and xanthan gum is present at a ratio of about 1:1 hydroxyethylcellulose:xanthan gum.

In some embodiments, the thickening agent, humectant (e.g., any of the humectants described herein), and first solvent are present in a ratio of about 1: 15:234 to about 1:62: 187 thickening agent:humectant:first solvent in the first solution. In some embodiments, the thickening agent and first solvent are present in a ratio of about 1:31:218 thickening agent:humectant:first solvent in the first solution.

In some embodiments, the humectant is glycerin.

In some embodiments, the first solution is incubated at a temperature of about 70° C. to about 120° C. For example, about 70° C. to about 75° C., about 70° C. to about 80° C., about 70° C. to about 85° C., about 70° C. to about 90° C., about 70° C. to about 95° C., about 70° C. to about 100° C., about 70° C. to about 105° C., about 70° C. to about 110° C., about 70° C. to about 115° C., about 115° C. to about 120° C., about 110° C. to about 120° C., about 105° C. to about 120° C., about 100° C. to about 120° C., about 95° C. to about 120° C., about 90° C. to about 120° C., about 85° C. to about 120° C., about 80° C. to about 120° C., or about 75° C. to about 120° C. In some embodiments, the first solution is incubated at a temperature of about 80° C. to about 105° C., about 85° C. to about 100° C., or about 90° C. to about 95° C.

In some embodiments, the first solution is stirred.

In some embodiments, the first solution is incubated and/or stirred until the thickening agent is dissolved.

In some embodiments, the genipin and second solvent are present in a ratio of about 4:25 to about 3:5 genipin:second solvent in the second solution. In some embodiments, the genipin and second solvent are present in a ratio of about 3:10 genipin:second solvent in the second solution.

In some embodiments, the genipin, first film-forming agent (e.g., any of the film-forming agents described herein), and second solvent are present in a ratio of about 57:16:177 to about 57:4:189 genipin:film-forming agent:second solvent in the second solution. In some embodiments, the genipin, first film-forming agent, and second solvent are present in a ratio of about 57:8: 185 genipin:film-forming agent:second solvent in the second solution. In some embodiments, the first film-forming agent is polymethylsilsesquioxane.

In some embodiments, the second solution is incubated at a temperature of about 30° C. to about 60° C. For example, about 30° C. to about 35° C., about 30° C. to about 40° C., about 30° C. to about 45° C., about 30° C. to about 50° C., about 30° C. to about 55° C., about 55° C. to about 60° C., about 50° C. to about 60° C., about 45° C. to about 60° C., about 40° C. to about 60° C., or about 35° C. to about 60° C. In some embodiments, the second solution is incubated at a temperature of about 35° C. to about 55° C., about 35° C. to about 45° C., about 40° C. to about 50° C., or about 45° C. to about 55° C.

In some embodiments, the second solution is stirred.

In some embodiments, the second solution is incubated and/or stirred until the genipin is dissolved.

In some embodiments, the temperature of the first solution is cooled. In some embodiments, the temperature of the first solution is cooled to about 20° C. to about 50° C. For example, about 20° C. to about 25° C., about 20° C. to about 30° C., about 20° C. to about 35° C., about 20° C. to about 40° C., about 20° C. about 45° C., about 45° C. to about 50° C., about 40° C. to about 50° C., about 35° C. to about 50° C., about 30° C. to about 50° C., or about 25° C. to about 50° C. In some embodiments, the temperature of the first solution is cooled to about 35° C. to about 45° C. For example, about 35° C., about 36° C., about 37° C., about 38° C., about 39° C., about 40° C., about 41° C., about 42° C., about 43° C., about 44° C., or about 45° C.

In some embodiments, the temperature of the second solution is cooled. In some embodiments, the temperature of the second solution is cooled to about 20° C. to about 50° C. For example, about 20° C. to about 25° C., about 20° C. to about 30° C., about 20° C. to about 35° C., about 20° C. to about 40° C., about 20° C. about 45° C., about 45° C. to about 50° C., about 40° C. to about 50° C., about 35° C. to about 50° C., about 30° C. to about 50° C., or about 25° C. to about 50° C. In some embodiments, the temperature of the second solution is cooled to about 35° C. to about 45° C. For example, about 35° C., about 36° C., about 37° C., about 38° C., about 39° C., about 40° C., about 41° C., about 42° C., about 43° C., about 44° C., or about 45° C.

In some embodiments, the first solution and second solution are present in a ratio of about 1:4 to about 1:1 first solution:second solution in the ink composition. In some embodiments, the first solution and second solution are present in a ratio of about 1:2 first solution: second solution in the ink composition.

In some embodiments, combining the first solution and second solution to form an ink composition includes stirring. In some embodiments, the ink composition is stirred until the ink composition is clear without any visible suspended particles.

In some embodiments, the ink composition is cooled to about room temperature. In some embodiments, the ink composition is cooled to about 20° C. to about 30° C. In some embodiments, the ink composition is cooled to about 25° C.

In some embodiments, the ink composition is cooled prior to the adding a film-forming agent (e.g., a first or second film-forming agent) agent and/or a preservative to the ink composition. In some embodiments, adding a film-forming agent (e.g., a first or second film-forming agent) agent and/or a preservative to the ink composition further comprises stirring the ink composition.

In some embodiments, the film-forming agent (e.g., any of the film-forming agents described herein) is present in an amount of about 1% to about 10% w/w of the ink composition. In some embodiments, the film-forming agent is present in an amount of about 2% to about 6% w/w of the ink composition. In some embodiments, the film-forming agent is selected from the group consisting of: polymethylsilsesquioxane, acrylates/C12-22 alkyl methacrylate copolymer, and a combination thereof.

In some embodiments, the second film-forming agent (e.g., any of the film-forming agents described herein) is present in an amount of about 1% to about 5% w/w of the ink composition. In some embodiments, the second film-forming agent is present in an amount of about 3% w/w of the ink composition. In some embodiments, the first film-forming agent and the second film-forming agent are different.

In some embodiments, the second film-forming agent is acrylates/C12-22 alkyl methacrylate copolymer.

In some embodiments, the preservative (e.g., any of the preservatives described herein) is present in an amount of about 0.1% to about 5% w/w of the ink composition. In some embodiments, the preservative is present in an amount of about 2% to about 4% w/w of the ink composition. In some embodiments, the preservative is present in an amount of about 3% w/w of the ink composition.

In some embodiments, the preservative comprises a combination of phenoxyethanol and ethylhexylglycerin. In some embodiments, phenoxyethanol and ethylhexylglycerin are present in the composition at a ratio of about 9:1 phenoxyethanol:ethylhexylglycerin. In some embodiments, the combination of phenoxyethanol and ethylhexylglycerin is present in an amount of about 0.1% to about 2% w/w of the ink composition. In some embodiments, the combination of phenoxyethanol and ethylhexylglycerin is present in an amount of about 1% w/w of the ink composition.

In some embodiments, the preservative comprises 1,2-hexanediol. In some embodiments, the 1,2-hexanediol is present in an amount of about 1% to about 3% w/w of the ink composition. In some embodiments, the 1,2-hexanediol is present in an amount of about 2% w/w of the ink composition.

In some embodiments, methods of manufacturing an ink composition as described herein include: i) combining genipin with a first solvent to form a genipin solution; ii) combining a film-forming agent (e.g., a first film-forming agent) with a second solvent to form a solution (e.g., a first solution or a subsequent solution); and iii) combining the genipin solution and a solution (e.g., a first solution or a subsequent solution) to form an ink composition. In some embodiments, the methods described herein can further include adding one or more of a film-forming agent (e.g., a second or third film-forming agent), a humectant (e.g., a first humectant), a preservative (e.g., a first preservative), and a temporary colorant (e.g., a first temporary colorant) to a genipin solution, a solution (e.g., a first solution or a subsequent solution), or the ink composition. In some embodiments, the methods described herein include adding one or more of a film-forming agent (e.g., a second or third film-forming agent), a humectant (e.g., a first humectant), and a preservative (e.g., a first preservative) to the solution (e.g., a first solution) to form a second solution (or a third solution, a fourth solution, etc., or a subsequent solution).

In some embodiments, the first solvent is any of the solvents described herein. In some embodiments, the second solvent is any of the solvents described herein. In some embodiments, the first solvent and second solvent are different.

In some embodiments, the first solvent comprises ethanol. In some embodiments, the first solvent comprises ethanol and water. In some embodiments, the second solvent comprises water.

In some embodiments, the genipin and first solvent are present in a ratio of about 4:25 to about 3:5 genipin:first solvent in the genipin solution. In some embodiments, the genipin and first solvent are present in a ratio of about 1:5 genipin:first solvent in the genipin solution.

In some embodiments, the genipin solution is stirred. In some embodiments, the genipin solution is stirred until the genipin is dissolved.

In some embodiments, the film-forming agent (e.g., a first film-forming agent) and second solvent are present in a ratio of about 1:40 to about 1:120 film-forming agent: second solvent in the solution. In some embodiments, the film-forming agent and second solvent are present in a ratio of about 1:80 film-forming agent:second solvent in the solution.

In some embodiments, the film-forming agent (e.g., a first film-forming agent) is any of the film-forming agents described herein. In some embodiments, the film-forming agent comprises acrylates copolymer.

In some embodiments, the solution (e.g., the first solution) is stirred. In some embodiments, the solution (e.g., the first solution) is stirred until the film-forming agent (e.g., a first film-forming agent) is dispersed.

In some embodiments, a second film-forming agent is combined with the first solution to form a second solution. In some embodiments, the second film-forming agent and first solution are present in a ratio of about 1:10 to about 1:40 second film-forming agent:first solution in the second solution. In some embodiments, the second film-forming agent and first solution are present in a ratio of about 1:21 second film-forming agent:first solution in the second solution.

In some embodiments, the second film-forming agent is any of the film-forming agents described herein. In some embodiments, the second film-forming agent comprises polyvinylpyrrolidone.

In some embodiments, the second solution is stirred.

In some embodiments, a third film-forming agent is combined with the second solution to form a third solution. In some embodiments, the third film-forming agent and second solution are present in a ratio of about 1:2 to about 1:10 third film-forming agent:second solution in the third solution. In some embodiments, the third film-forming agent and second solution are present in a ratio of about 3:11 third film-forming agent:second solution in the third solution.

In some embodiments, the third film-forming agent is any of the film-forming agents described herein. In some embodiments, the third film-forming agent comprises styrene/acrylates copolymer.

In some embodiments, the third solution is stirred.

In some embodiments, a humectant is combined with a solution (e.g., a first solution, a second solution, or a third solution) to form a fourth solution. In some embodiments, a humectant is combined with the third solution to form a fourth solution. In some embodiments, the humectant and solution (e.g., third solution) are present in a ratio of about 1:10 to about 1:40 humectant:solution in the fourth solution. In some embodiments, the humectant and solution (e.g., third solution) are present in a ratio of about 1:28 humectant:solution in the fourth solution.

In some embodiments, the humectant is any of the humectants described herein. In some embodiments, the humectant comprises propanediol.

In some embodiments, the fourth solution is stirred.

In some embodiments, a preservative is combined with a solution (e.g., a first solution, a second solution, a third solution, or a fourth solution) to form a fifth solution. In some embodiments, a preservative is combined with the fourth solution to form a fifth solution. In some embodiments, the preservative and solution (e.g., fourth solution) are present in a ratio of about 1:10 to about 1:40 preservative:solution in the fifth solution. In some embodiments, the preservative and solution (e.g., fourth solution) are present in a ratio of about 1:28 preservative: solution in the fifth solution.

In some embodiments, the preservative is any of the preservatives described herein. In some embodiments, the preservative comprises a combination of phenoxyethanol and ethylhexylglycerin. In some embodiments, the preservative comprises a combination of phenoxyethanol ethylhexylglycerin, and EDTA. In some embodiments, the phenoxyethanol and ethylhexylglycerin are combined with the solution (e.g., a fourth solution) before the EDTA is combined with the solution. In some embodiments, the solution (e.g., the fourth solution) is stirred between combining the phenoxyethanol and ethylhexylglycerin with the solution (e.g., the fourth solution) and combining the EDTA with the solution (e.g., the fourth solution).

In some embodiments, phenoxyethanol and ethylhexylglycerin are present in the composition at a ratio of about 9:1 phenoxyethanol:ethylhexylglycerin.

In some embodiments, the fifth solution is stirred.

In some embodiments, the genipin solution and solution (e.g., a first solution, a second solution, a third solution, a fourth solution, or a fifth solution) are present in a ratio of about 1:6 to about 1:1 genipin solution:solution in the ink composition. In some embodiments, the genipin solution and solution (e.g., a first solution, a second solution, a third solution, a fourth solution, or a fifth solution) are present in a ratio of about 1:2 genipin solution: solution in the ink composition.

In some embodiments, combining the genipin solution and second solution to form an ink composition includes stirring. In some embodiments, the ink composition is stirred until the ink composition is clear without any visible suspended particles.

In some embodiments, a temporary colorant (e.g., any of the temporary colorants described herein) is combined with the ink composition. In some embodiments, the temporary colorant is present in an amount of about 0.1% to about 10% w/w of the ink composition. In some embodiments, the temporary colorant is present in an amount of about 2.5% w/w of the ink composition. In some embodiments, the temporary colorant comprises D&C Black No. 2.

Also provided herein are compositions prepared by any of the methods described above.

### EXAMPLES

The invention will be described in greater detail by way of specific examples. The following examples are offered for illustrative purposes, and to exemplify the topical compositions and methods described herein and are not intended to limit the invention in any manner. Many variations will suggest themselves and are within the full-intended scope. Those of skill in the art will readily recognize a variety of non-critical parameters that can be changed or modified to yield essentially the same results.

### Example 1. Ink Compositions

0.03 g hydroexthylcellulose (e.g., NATROSOL® 250 HHR CS), 0.03 g xanthan gum (e.g., KELTROL® CG), and 2 g glycerol were added to 14.2 g water (Phase A) and mixed at a temperature of about 90° C. to 95° C. until complete dissolution of the hydroexthylcellulose and xanthan gum. The temperature was subsequently lowered to 40° C.

7 g genipin (e.g., Genipa plant extract) and 1 g polymethylsilsesquioxane (e.g., GRANSIL™ PSQ) were added to 22.8 g ethanol (Phase B) and mixed at a temperature of about 40° C. to 50° C. Degradation of genipin should be avoided. After complete suspension of genipin in the solution, the temperature was lowered to 40° C.

Phases A and B were mixed until the solution was clear and without any visible suspended particles. The ink mixture was then brought to room temperature.

1.5 g acrylates/C12-22 alkyl methacrylate copolymer (e.g,. ALLIANZ™ OPT polymer), 0.5 g of a combination of phenoxyethanol and ethylhexylglycerin (e.g,. EUXYL® PE9010), and 1 g 1,2-hexanediol (e.g., OPTIPHEN® HD) were added to the ink mixture and mixed until a uniform solution was achieved. The final amounts were as shown in Table 1.

TABLE 1

Ink composition

| | Ingredient | Amount (% w/w) | Amount (g) |
|---|---|---|---|
| Phase A | Aqua | 28.4 | 14.2 |
| | Glycerol | 4.0 | 2 |
| | Hydroexthylcellulose | 0.06 | 0.03 |
| | Xanthan Gum | 0.06 | 0.03 |
| Phase B | Ethanol | 45.5 | 22.8 |
| | Genipa Plant Extract | 14.0 | 7 |
| | Polymethylsilsesquioxane | 2.0 | 1 |
| Additions | Acrylates/C 12-22 Alkyl Methacrylate Copolymer | 3.0 | 1.5 |
| | Phenoxyethanol and) Ethylhexylglycerin | 1.0 | 0.5 |
| | 1,2-Hexanediol | 2.0 | 1 |

### Example 2. Ink Compositions

Genipin and ethanol were mixed together until the genipin was dissolved and a clear amber colored solution was obtained (Phase A). The solution was stirred at 500 rpm on a IKA mixer for 15 minutes.

Acrylates copolymer (e.g., CARBOPOL® Aqua SF1) was added to the water (Phase B). The mixture was mixed for 5-7 minutes at 500 rpm.

Phase C was added to Phase B under continuous mixing and until the powder was dispersed. This mixture was stirred at 500 rpm for 5-7 minutes.

Phase D was added to Phase B+C under continuous mixing and until the mixture was homogeneous. The Phase B+C+D mixture was stirred at 500 rpm for 5-7 minutes.

Phase E was added to Phase B+C+D under continuous mixing and until the mixture was homogeneous. The Phase B+C+D+E mixture was stirred at 500 rpm for 5-7 minutes.

Phase F was added to Phase B+C+D+E under continuous mixing and until the mixture was homogeneous. The Phase B+C+D+E+F mixture was stirred at 500 rpm for 5-7 minutes.

Phase G was added to Phase B+C+D+E+F under continuous mixing and until the mixture was homogeneous. The Phase B+C+D+E+F+G mixture was stirred at 500 rpm for 5-7 minutes.

Phase B+C+D+E+F+G was added to Phase A under continuous mixing and until the mixture was homogeneous. The Phase B+C+D+E+F+G+A mixture was stirred at 500 rpm for 10-15 minutes.

Phase H was added to Phase B+C+D+E+F+G+A. The Phase B+C+D+E+F+G+A+H mixture was stirred at 800-1000 rpm for 10 minutes until mixture was homogeneous.

| | Ingredient | Amount (% w/w) |
|---|---|---|
| Phase A | Genipin (Genipa Plant Extract) | 10 |
| | Ethanol 95% | 50 |
| Phase B | Water (Aqua) | 20.7 |
| | Acrylates Copolymer (e.g., CARBOPOL AQUA® SF1) | 0.25 |
| Phase C | Polyvinylpyrrolidone (e.g., PVP K-15) | 1 |
| Phase D | Styrene/Acrylates Copolymer (e.g., DAITOSOL 5000 STY) | 6 |
| Phase E | Propanediol (e.g., ZEMEA®) | 1 |
| Phase F | Disodium EDTA | 0.05 |
| Phase G | Phenoxyethanol (and) Ethylhexylglycerin (e.g., EUXYL® 9010 PE) | 1 |
| Phase H | Water, black 2, Sodium lignosulfonate (e.g., W25CB) | 10 |

### OTHER EMBODIMENTS

Embodiment 1 is a composition comprising: a semi-permanent colorant; a solvent; and a thickening agent.

Embodiment 2 is the composition of embodiment 1, wherein the semi-permanent colorant is present in an amount of about 10% to about 20% w/w of the composition.

Embodiment 3 is the composition of embodiment 1 or 2, wherein the semi-permanent colorant is present in an amount of about 12% to about 16% w/w of the composition.

Embodiment 4 is the composition of any one of embodiments 1-3, wherein the semi-permanent colorant is present in an amount of about 14% w/w of the composition.

Embodiment 5 is the composition of any one of embodiments 1-4, wherein the semi-permanent colorant is genipin.

Embodiment 6 is the composition of any one of embodiments 1-5, wherein the solvent is present in an amount of about 65% to about 80% w/w of the composition.

Embodiment 7 is the composition of any one of embodiments 1-6, wherein the solvent is present in an amount of about 72% to about 76% w/w of the composition.

Embodiment 8 is the composition of any one of embodiments 1-7, wherein the solvent is present in an amount of about 74% w/w of the composition.

Embodiment 9 is the composition of any one of embodiments 1-8, wherein the solvent comprises water, methanol, ethanol, isopropanol, diethylene glycol monoethyl ether, or a combination thereof.

Embodiment 10 is the composition of any one of embodiments 1-9, wherein the solvent is a combination of water and ethanol.

Embodiment 11 is the composition of any one of embodiments 1-10, wherein the water is present in an amount of about 25% to about 30% w/w of the composition.

Embodiment 12 is the composition of any one of embodiments 1-11, wherein the water is present in an amount of about 28% w/w of the composition.

Embodiment 13 is the composition of any one of embodiments 1-12, wherein the ethanol is present in an amount of about 40% to about 50% w/w of the composition.

Embodiment 14 is the composition of any one of embodiments 1-13, wherein the ethanol is present in an amount of about 45% to about 47% w/w of the composition.

Embodiment 15 is the composition of any one of embodiments 1-14, wherein the ethanol is present in an amount of about 46% w/w of the composition.

Embodiment 16 is the composition of any one of embodiments 1-15, wherein the thickening agent is present in an amount of about 0.01% to about 0.3% w/w of the composition.

Embodiment 17 is the composition of any one of embodiments 1-16, wherein the thickening agent is present in an amount of about 0.05% to about 0.2% w/w of the composition.

Embodiment 18 is the composition of any one of embodiments 1-17, wherein the thickening agent is present in an amount of about 0.12% w/w of the composition.

Embodiment 19 is the composition of any one of embodiments 1-18, wherein the thickening agent comprises hydroxyethylcellulose, xanthan gum, arabinogalactan, aluminum starch octenylsuccinate, xanthan gum, sclerotium gum, tragacanth gum, pectin, gum karaya, gum arabic, agar, guar gum, carrageenan, locust bean gum, alginate, alginin, gelatin, tara gum, gum ghatti, gellan gum, konjac gum, cassia gum, spruce gum, chicle gum, dammar, curdlan gum, pullulan, hydroxypropyl cellulose, hydroxypropyl methylcellulose, ethylcellulose, soybean hemicellulose, and sodium carboxy methylcellulose, or a combination thereof.

Embodiment 20 is the composition of any one of embodiments 1-19, wherein the thickening agent is a combination of hydroxyethylcellulose and xanthan gum.

Embodiment 21 is the composition of any one of embodiment 19 or 20, wherein the hydroxyethylcellulose is present in an amount of about 0.01% to about 0.1% w/w of the composition.

Embodiment 22 is the composition of any one of embodiments 19-21, wherein the hydroxyethylcellulose is present in an amount of about 0.04% to about 0.08% w/w of the composition.

Embodiment 23 is the composition of any one of embodiments 19-22, wherein the hydroxyethylcellulose is present in an amount of about 0.06% w/w of the composition.

Embodiment 24 is the composition of any one of embodiments 19-23, wherein the xanthan gum is present in an amount of about 0.01% to about 0.1% w/w of the composition.

Embodiment 25 is the composition of any one of embodiments 19-24, wherein the xanthan gum is present in an amount of about 0.04% to about 0.08% w/w of the composition.

Embodiment 26 is the composition of any one of embodiments 19-25, wherein the xanthan gum is present in an amount of about 0.06% w/w of the composition.

Embodiment 27 is the composition of any one of embodiments 1-26, wherein the composition further comprises a film-forming agent.

Embodiment 28 is the composition of any one of embodiments 1-27, wherein the film-forming agent is present in an amount of about 0.1% to about 10% w/w of the composition.

Embodiment 29 is the composition of any one of embodiments 1-28, wherein the film-forming agent is present in an amount of about 4% to about 6% w/w of the composition.

Embodiment 30 is the composition of any one of embodiments 1-29, wherein the film-forming agent is present in an amount of about 5% w/w of the composition.

Embodiment 31 is the composition of any one of embodiments 1-30, wherein the film-forming agent comprises a volatile silicon resin, polyvinylpyrrolidone, an acrylate, an acrylamide, a copolymer, an isododecane resin, or a combination thereof.

Embodiment 32 is the composition of any one of embodiments 1-31, wherein the film-forming agent comprises polymethylsilsesquioxane, trimethylsiloxysilicate, polypropylsilsesquioxane, dimethicone, cyclopentasiloxane, dimethiconol crosspolymer, polysilicone-6, polysilicone-8, polysilicone-11, polysilicone-14, acrylates/C 12-22 alkyl methacrylate copolymer, acrylates/polytrimethylsiloxymethacrylate copolymer, polyvinylpyrrolidone/vinyl acetate (VP/VA) copolymer, VP/dimethiconylacrylate/polycarbamyl/polyglycol ester, VP/dimethylaminoethylmethacrylate copolymer, VP/dimethylamino ethylmethacrylate/polycarbamyl polyglycol ester, VP/eicosene copolymer, VP/hexadecene copolymer, VP/methacrylamide/vinyl imidazole copolymer, VP/polycarbamyl polyglycol ester, VP/VA copolymer, polyester-1, polyester-2, polyester-3, polyester-4, polyester-5, polyester-7, polyester-8, polyester-10 or a combination thereof.

Embodiment 33 is the composition of any one of embodiments 1-32, wherein the film-forming agent is a combination of polymethylsilsesquioxane and acrylates/C12-22 alkyl methacrylate copolymer.

Embodiment 34 is the composition of embodiment 32 or 33, wherein the polymethylsilsesquioxane is present in an amount of about 1% to about 5% w/w of the composition.

Embodiment 35 is the composition of any one of embodiments 32-34, wherein the polymethylsilsesquioxane is present in an amount of about 2% w/w of the composition.

Embodiment 36 is the composition of any one of embodiments 32-35, wherein the acrylates/C12-22 alkyl methacrylate copolymer is present in an amount of about 1% to about 5% w/w of the composition.

Embodiment 37 is the composition of any one of embodiments 32-36, wherein the acrylates/C12-22 alkyl methacrylate copolymer is present in an amount of about 3% w/w of the composition.

Embodiment 38 is the composition of any one of embodiments 1-37, wherein the composition further comprises a humectant.

Embodiment 39 is the composition of any one of embodiments 1-38, wherein the humectant is present in an amount of about 1% to about 10% w/w of the composition.

Embodiment 40 is the composition of any one of embodiments 1-39, wherein the humectant is present in an amount of about 2% to about 6% w/w of the composition.

Embodiment 41 is the composition of any one of embodiments 1-40, wherein the humectant is present in an amount of about 4% w/w of the composition.

Embodiment 42 is the composition of any one of embodiments 1-41, wherein the humectant comprises Aloe vera gel, lithium chloride, allantoin, urea, dicyanamideglycerol, lactic acid, propylene glycol, polyethylene glycol, dipropylene glycol, polypropylene glycol, polyethylene glycol and derivatives thereof, hexylene glycol, 1,3-butylene glycol, 1,2,6-hexanetriol, ethoxylated glycerol, propoxylated glycerol, glyceryl triacetate, sorbitol, hydroxypropyl sorbitol, xylitol, maltitol, hyaluronic acid, Tremella extract, sodium lactate, sodium L-pyroglutamate urea, and pyrrolidone carboxylic acid, or a combination thereof.

Embodiment 43 is the composition of any one of embodiments 1-42, wherein the humectant is glycerin.

Embodiment 44 is the composition of any one of embodiments 1-43, wherein the composition further comprises a preservative.

Embodiment 45 is the composition of embodiment 44, wherein the preservative is present in an amount of about 0.1% to about 5% w/w of the composition.

Embodiment 46 is the composition of embodiment 44 or 45, wherein the preservative is present in an amount of about 2% to about 4% w/w of the composition.

Embodiment 47 is the composition of any one of embodiment 44-46, wherein the preservative is present in an amount of about 3% w/w of the composition.

Embodiment 48 is the composition of any one of embodiment 44-47, wherein the preservative comprises ascorbic acid, an ascorbate, a palmitate, citric acid, a benzoate, a benzoic acid, a propionate, propionic acid, a sorbate, sorbic acid, a salicylic acid, a salicylate, hexa-2,4-dienoic acid, a hexa-2,4-dienoate, formaldehyde, a formaldehyde releaser, formic acid and its salts, 3-acetyl-6-methylpyran-2,4-(3H)-dione and its salts, 3,3′-dibromo-4,4′- hexamethylenedioxydibenzamidine and its salts, thiomersal, phenylmercuric salts, undec-10-enoic acid and its salts, 1,3-bis (2-ethylhexyl) hexahydro-5-methyl-5-pyrimidine, 5-bromo-5-nitro-1,3-dioxane, bronopol, 2,4-dichlorobenzyl alcohol, 1-(4-chlorophenyl)-3-(3,4-dichlorophenyl) urea, chlorocresol, chloroxylenol, 5-chloro-2-(2,4- dichlorophenoxy) phenol, N,N″-methylenebis[N′-[3-(hydroxymethyl)-2,5- dioxoimidazolidin-4-yl]urea], polyaminopropyl biguanide, methenamine, quaternium-15, climbazole, DMDM hydantoin, benzyl alcohol, 1-Hydroxy-4-methyl-6-(2,4,4-trimethylpentyl)-2-pyridon, piroctone olamine, bromochlorophene, o-cymen-5-ol, chlorophene, chloroacetaminde, methylchloroisothiazolinone, methylisothiazolinone, phenoxyisopropanol, chlorhexidine, chlorhexidine diacetate, chlorhexidine digluconate, chlorhexidine dihydrochloride, dimethyl oxazolidine, behentrimonium chloride, cetri-monium bromide, cetrimonium chloride, laurtrimonium bromide, laurtrimonium chloride, steartrimonium bromide, steartrimonium chloride, diazolidinyl urea, hexamidine, hexamidine diisethionate, hexamidine paraben, glutaral, 7-ethylbicyclooxazolidine, chlorphenesin, sodium hydroxymethylglycinate, silver chloride, benzethonium chloride, benzalkonium chloride, benzalkonium bromide, benzalkonium saccharinate, benzylhemiformal, iodopropynyl butylcarbamate, biphenyl-2-ol and its salts, pyrithionine zinc, an erythorbate, a nitrite, ethylenediaminetetraacetic acid (EDTA), butylated hydroxyanisole (BHA), butylated hydroxytoluene (BHT), capryllic acid, dilauryl thiodipropionate, erythorbic acid, gum guaiac, methylparaben, a sulfite, a bisulfite, a metabisulfite, propyl gallatepy, propylparaben, stannous chloride, sulfur dioxide, thiodipropionic acid, an isothiazoline, a paraben, phenoxyethanol, ethylhexylglycerin, a glycol, a tocopherol, 1,2-hexanediol, or a combination thereof.

Embodiment 49 is the composition of any one of embodiment 44-48, wherein the preservative comprises a combination of phenoxyethanol and ethylhexylglycerin.

Embodiment 50 is the composition of embodiment 49, wherein the phenoxyethanol and ethylhexylglycerin are present in the composition at a ratio of about 9:1 phenoxyethanol:ethylhexylglycerin.

Embodiment 51 is the composition of embodiment 49 or 50, wherein the combination of phenoxyethanol and ethylhexylglycerin is present in an amount of about 0.1% to about 2% w/w of the composition.

Embodiment 52 is the composition of any one of embodiments 49-51, wherein the combination of phenoxyethanol and ethylhexylglycerin is present in an amount of about 1% w/w of the composition.

Embodiment 53 is the composition of any one of embodiments 49-52, wherein the preservative comprises 1,2-hexanediol.

Embodiment 54 is the composition of any one of embodiments 49-53, wherein the 1,2-hexanediol is present in an amount of about 1% to about 3% w/w of the composition.

Embodiment 55 is the composition of any one of embodiments 49-54, wherein the 1,2-hexanediol is present in an amount of about 2% w/w of the composition.

Embodiment 56 is a method of manufacturing an ink composition, the method comprising: i. combining a thickening agent with a first solvent to form a first solution; ii. combining genipin with a second solvent to form a second solution; and iii. combining the first solution and second solution to form the ink composition.

Embodiment 57 is the method of embodiment 56, wherein the method further comprises adding a film-forming agent, a preservative, or a combination thereof to the ink composition.

Embodiment 58 is a method of manufacturing an ink composition, the method comprising: i. combining a thickening agent and a humectant with a first solvent to form a first solution; ii. combining genipin and a first film-forming agent with a second solvent to form a second solution; and iii. combining the first solution and second solution to form the ink composition.

Embodiment 59 is the method of embodiment 58, wherein the method further comprises adding a second film-forming agent, a preservative, or a combination thereof to the ink composition.

Embodiment 60 is the method of any one of embodiments 56-59, wherein the first solution is incubated at a temperature of about 70° C. to about 120° C.

Embodiment 61 is the method of any one of embodiments 56-59, wherein the first solution is incubated at a temperature of about 90° C. to about 95° C.

Embodiment 62 is the method of any one of embodiments 56-61, wherein the first solution is stirred.

Embodiment 63 is the method of any one of embodiments 56-62, wherein the thickening agent is dissolved in the first solution prior to combining the first solution and second solution.

Embodiment 64 is the method of any one of embodiments 56-63, wherein the second solution is incubated at a temperature of about 30° C. to about 60° C.

Embodiment 65 is the method of any one of embodiments 56-64, wherein the second solution is incubated at a temperature of about 40° C. to about 50° C.

Embodiment 66 is the method of any one of embodiments 56-65, wherein the second solution is stirred.

Embodiment 67 is the method any one of embodiments 60-66, wherein the first solution is cooled to a temperature of about 20° C. to about 50° C.

Embodiment 68 is the method any one of embodiments 60-67, wherein the first solution is cooled to a temperature of about 40° C.

Embodiment 69 is the method any one of embodiments 65-68, wherein the second solution is cooled to a temperature of about 20° C. to about 50° C.

Embodiment 70 is the method any one of embodiments 65-69, wherein the first solution is cooled to a temperature of about 40° C.

Embodiment 71 is the method of any one of embodiments 5670, wherein the ink composition is stirred.

Embodiment 72 is the method any one of embodiments 65-68, wherein the ink composition is cooled to a temperature of about 20° C. to about 30° C.

Embodiment 73 is the method any one of embodiments 65-69, wherein the ink composition is cooled to a temperature of about 25° C.

Embodiment 74 is the method of embodiment 72 or 73, wherein the ink composition is cooled prior to adding the film-forming agent, preservative, or combination thereof to the ink composition.

Embodiment 75 is the method of any one of embodiments 55-74, wherein the thickening agent and first solvent are present in a ratio of about 1:500 to about 4:500 thickening agent:first solvent in the first solution.

Embodiment 76 is the method of any one of embodiments 56-75, wherein the thickening agent and first solvent are present in a ratio of about 2:500 thickening agent:first solvent in the first solution.

Embodiment 77 is the method of any one of embodiments 56-75, wherein the first solvent comprises water, methanol, ethanol, isopropanol, diethylene glycol monoethyl ether, or a combination thereof.

Embodiment 78 is the method of any one of embodiments 56-75, wherein the first solvent comprises water.

Embodiment 79 is the method of any one of embodiments 56-78, wherein the thickening agent comprises hydroxyethylcellulose, xanthan gum, arabinogalactan, aluminum starch octenylsuccinate, xanthan gum, sclerotium gum, tragacanth gum, pectin, gum karaya, gum arabic, agar, guar gum, carrageenan, locust bean gum, alginate, alginin, gelatin, tara gum, gum ghatti, gellan gum, konjac gum, cassia gum, spruce gum, chicle gum, dammar, curdlan gum, pullulan, hydroxypropyl cellulose, hydroxypropyl methylcellulose, ethylcellulose, soybean hemicellulose, and sodium carboxy methylcellulose, or a combination thereof.

Embodiment 80 is the method of any one of embodiments 56-79, wherein the thickening agent is a combination of hydroxyethylcellulose and xanthan gum.

Embodiment 81 is the method of embodiment 80, wherein the combination of hydroxyethylcellulose and xanthan gum is present at a ratio of 1:1 hydroxyethylcellulose:xanthan gum.

Embodiment 82 is the method of any one of embodiments 58-81, wherein the thickening agent, humectant, and first solvent are present in a ratio of about 1:15:234 to about 1:62:187 thickening agent:humectant:first solvent in the first solution.

Embodiment 83 is the method of any one of embodiments 58-82, wherein the thickening agent and first solvent are present in a ratio of about 1:31:218 thickening agent:humectant:first solvent in the first solution.

Embodiment 84 is the method of any one of embodiments 58-83, wherein the humectant comprises Aloe vera gel, lithium chloride, allantoin, urea, dicyanamideglycerol, lactic acid, propylene glycol, polyethylene glycol, dipropylene glycol, polypropylene glycol, polyethylene glycol and derivatives thereof, hexylene glycol, 1,3-butylene glycol, 1,2,6-hexanetriol, ethoxylated glycerol, propoxylated glycerol, glyceryl triacetate, sorbitol, hydroxypropyl sorbitol, xylitol, maltitol, hyaluronic acid, Tremella extract, sodium lactate, sodium L-pyroglutamate urea, and pyrrolidone carboxylic acid, or a combination thereof.

Embodiment 85 is the method of any one of embodiments 58-84, wherein the humectant is glycerin.

Embodiment 86 is the method of any one of embodiments 56-85, wherein the genipin and second solvent are present in a ratio of about 4:25 to about 3:5 genipin: second solvent in the second solution.

Embodiment 87 is the method of any one of embodiments 56-86, wherein the genipin and second solvent are present in a ratio of about 3:10 genipin:second solvent in the second solution.

Embodiment 88 is the method of any one of embodiments 56-87, wherein the second solvent comprises water, methanol, ethanol, isopropanol, diethylene glycol monoethyl ether, or a combination thereof.

Embodiment 89 is the method of any one of embodiments 56-88, wherein the second solvent comprises ethanol.

Embodiment 90 is the method of any one of embodiments 58-89, wherein the genipin, first film-forming agent, and second solvent are present in a ratio of about 57:16:177 to about 57:4:189 genipin:film-forming agent:second solvent in the second solution.

Embodiment 91 is the method of any one of embodiments 58-90, wherein the genipin, first film-forming agent, and second solvent are present in a ratio of about 57:8:185 genipin:film-forming agent:second solvent in the second solution.

Embodiment 92 is the method of any one of embodiments 58-91, wherein the first film-forming agent comprises polymethylsilsesquioxane, trimethylsiloxysilicate, polypropylsilsesquioxane, dimethicone, cyclopentasiloxane, dimethiconol crosspolymer, polysilicone-6, polysilicone-8, polysilicone-11, polysilicone-14, acrylates/C12-22 alkyl methacrylate copolymer, acrylates/polytrimethylsiloxymethacrylate copolymer, polyvinylpyrrolidone/vinyl acetate (VP/VA) copolymer, VP/dimethiconylacrylate/polycarbamyl/polyglycol ester, VP/dimethylaminoethylmethacrylate copolymer, VP/dimethylamino ethylmethacrylate/polycarbamyl polyglycol ester, VP/eicosene copolymer, VP/hexadecene copolymer, VP/methacrylamide/vinyl imidazole copolymer, VP/polycarbamyl polyglycol ester, VP/VA copolymer, polyester-1, polyester-2, polyester-3, polyester-4, polyester-5, polyester-7, polyester-8, polyester-10 or a combination thereof.

Embodiment 93 is the method of embodiment 92, wherein the first film-forming agent is polymethylsilsesquioxane.

Embodiment 94 is the method of any one of embodiments 56-93, wherein the first solution and second solution are present in a ratio of about 1:4 to about 1:1 first solution: second solution in the ink composition.

Embodiment 95 is the method of any one of embodiments 56-94, wherein the first solution and second solution are present in a ratio of about 1:2 first solution: second solution in the ink composition.

Embodiment 96 is the method of any one of embodiments 57 and 60-95, wherein the film-forming agent is present in an amount of about 1% to about 10% w/w of the ink composition.

Embodiment 97 is the method of any one of embodiments 57 and 60-96, wherein the film-forming agent is present in an amount of about 2% to about 6% w/w of the ink composition.

Embodiment 98 is the method of any one of embodiments 57 and 60-97, wherein the film-forming agent comprises a volatile silicon resin, polyvinylpyrrolidone, an acrylate, an acrylamide, a copolymer, an isododecane resin, or a combination thereof.

Embodiment 99 is the method of any one of embodiments 57 and 60-98, wherein the film-forming agent comprises polymethylsilsesquioxane, trimethylsiloxysilicate, polypropylsilsesquioxane, dimethicone, cyclopentasiloxane, dimethiconol crosspolymer, polysilicone-6, polysilicone-8, polysilicone-11, polysilicone-14, acrylates/C 12-22 alkyl methacrylate copolymer, acrylates/polytrimethylsiloxymethacrylate copolymer, polyvinylpyrrolidone/vinyl acetate (VP/VA) copolymer, VP/dimethiconylacrylate/polycarbamyl/polyglycol ester, VP/dimethylaminoethylmethacrylate copolymer, VP/dimethylamino ethylmethacrylate/polycarbamyl polyglycol ester, VP/eicosene copolymer, VP/hexadecene copolymer, VP/methacrylamide/vinyl imidazole copolymer, VP/polycarbamyl polyglycol ester, VP/VA copolymer, polyester-1, polyester-2, polyester-3, polyester-4, polyester-5, polyester-7, polyester-8, polyester-10 or a combination thereof.

Embodiment 100 is the method of any one of embodiments 57 and 60-99, wherein the film-forming agent is selected from the group consisting of: polymethylsilsesquioxane, acrylates/C12-22 alkyl methacrylate copolymer, and a combination thereof.

Embodiment 101 is the method of any one of embodiments 59-95, wherein the second film-forming agent is present in an amount of about 1% to about 5% w/w of the ink composition.

Embodiment 102 is the method of any one of embodiments 59-101, wherein the second film-forming agent is present in an amount of about 3% w/w of the ink composition.

Embodiment 103 is the method of any one of embodiments 59-102, wherein the second film-forming agent comprises polymethylsilsesquioxane, trimethylsiloxysilicate, polypropylsilsesquioxane, dimethicone, cyclopentasiloxane, dimethiconol crosspolymer, polysilicone-6, polysilicone-8, polysilicone-11, polysilicone-14, acrylates/C12-22 alkyl methacrylate copolymer, acrylates/polytrimethylsiloxymethacrylate copolymer, polyvinylpyrrolidone/vinyl acetate (VP/VA) copolymer, VP/dimethiconylacrylate/polycarbamyl/polyglycol ester, VP/dimethylaminoethylmethacrylate copolymer, VP/dimethylamino ethylmethacrylate/polycarbamyl polyglycol ester, VP/eicosene copolymer, VP/hexadecene copolymer, VP/methacrylamide/vinyl imidazole copolymer, VP/polycarbamyl polyglycol ester, VP/VA copolymer, polyester-1, polyester-2, polyester-3, polyester-4, polyester-5, polyester-7, polyester-8, polyester-10 or a combination thereof.

Embodiment 104 is the method of any one of embodiments 59-103, wherein the second film-forming agent is acrylates/C12-22 alkyl methacrylate copolymer.

Embodiment 105 is the method of any one of embodiments 57 and 59-104, wherein the preservative is present in an amount of about 0.1% to about 5% w/w of the ink composition.

Embodiment 106 is the method of any one of embodiments 57 and 59-105, wherein the preservative is present in an amount of about 2% to about 4% w/w of the ink composition.

Embodiment 107 is the method of any one of embodiments 57 and 59-106, wherein the preservative is present in an amount of about 3% w/w of the ink composition.

Embodiment 108 is the method of any one of embodiments 57 and 59-107, wherein the preservative comprises ascorbic acid, an ascorbate, a palmitate, citric acid, a benzoate, a benzoic acid, a propionate, propionic acid, a sorbate, sorbic acid, a salicylic acid, a salicylate, hexa-2,4-dienoic acid, a hexa-2,4-dienoate, formaldehyde, a formaldehyde releaser, formic acid and its salts, 3-acetyl-6-methylpyran-2,4-(3H)-dione and its salts, 3,3′-dibromo-4,4′- hexamethylenedioxydibenzamidine and its salts, thiomersal, phenylmercuric salts, undec-10-enoic acid and its salts, 1,3-bis (2-ethylhexyl) hexahydro-5-methyl-5-pyrimidine, 5-bromo-5-nitro-1,3-dioxane, bronopol, 2,4-dichlorobenzyl alcohol, 1-(4-chlorophenyl)-3-(3,4-dichlorophenyl) urea, chlorocresol, chloroxylenol, 5-chloro-2-(2,4- dichlorophenoxy) phenol, N,N″-methylenebis[N′-[3-(hydroxymethyl)-2,5- dioxoimidazolidin-4-yl]urea], polyaminopropyl biguanide, methenamine, quaternium-15, climbazole, DMDM hydantoin, benzyl alcohol, 1-Hydroxy-4-methyl-6-(2,4,4-trimethylpentyl)-2-pyridon, piroctone olamine, bromochlorophene, o-cymen-5-ol, chlorophene, chloroacetaminde, methylchloroisothiazolinone, methylisothiazolinone, phenoxyisopropanol, chlorhexidine, chlorhexidine diacetate, chlorhexidine digluconate, chlorhexidine dihydrochloride, dimethyl oxazolidine, behentrimonium chloride, cetri-monium bromide, cetrimonium chloride, laurtrimonium bromide, laurtrimonium chloride, steartrimonium bromide, steartrimonium chloride, diazolidinyl urea, hexamidine, hexamidine diisethionate, hexamidine paraben, glutaral, 7-ethylbicyclooxazolidine, chlorphenesin, sodium hydroxymethylglycinate, silver chloride, benzethonium chloride, benzalkonium chloride, benzalkonium bromide, benzalkonium saccharinate, benzylhemiformal, iodopropynyl butylcarbamate, biphenyl-2-ol and its salts, pyrithionine zinc, an erythorbate, a nitrite, ethylenediaminetetraacetic acid (EDTA), butylated hydroxyanisole (BHA), butylated hydroxytoluene (BHT), capryllic acid, dilauryl thiodipropionate, erythorbic acid, gum guaiac, methylparaben, a sulfite, a bisulfite, a metabisulfite, propyl gallatepy, propylparaben, stannous chloride, sulfur dioxide, thiodipropionic acid, an isothiazoline, a paraben, phenoxyethanol, ethylhexylglycerin, a glycol, a tocopherol, 1,2-hexanediol, or a combination thereof.

Embodiment 109 is the method of any one of embodiments 57 and 59-108, wherein the preservative comprises a combination of phenoxyethanol and ethylhexylglycerin.

Embodiment 110 is the method of any one of embodiments 57 and 59-109, wherein the phenoxyethanol and ethylhexylglycerin are present in the composition at a ratio of about 9:1 phenoxyethanol:ethylhexylglycerin.

Embodiment 111 is the method of any one of embodiments 57 and 59-110, wherein the combination of phenoxyethanol and ethylhexylglycerin is present in an amount of about 0.1% to about 2% w/w of the ink composition.

Embodiment 112 is the method of any one of embodiments 57 and 59-111, wherein the combination of phenoxyethanol and ethylhexylglycerin is present in an amount of about 1% w/w of the ink composition.

Embodiment 113 is the method of any one of embodiments 57 and 59-112, wherein the preservative comprises 1,2-hexanediol.

Embodiment 114 is the method of any one of embodiments 57 and 59-113, wherein the 1,2-hexanediol is present in an amount of about 1% to about 3% w/w of the ink composition.

Embodiment 115 is the method of any one of embodiments 57 and 59-114, wherein the 1,2-hexanediol is present in an amount of about 2% w/w of the ink composition.

Embodiment 116 is an ink composition prepared by the methods of any one of embodiments 56-115.

It is to be understood that while the invention has been described in conjunction with the detailed description thereof, the foregoing description is intended to illustrate and not limit the scope of the invention which is defined by the scope of the appended claims. Other aspects, advantages, and modification are within the scope of the following claims.

1. A composition comprising:
a semi-permanent colorant;
a solvent; and
a film-forming agent.

2. The composition of claim 1, wherein the semi-permanent colorant is present in an amount of about 5% to about 15% w/w of the composition.

3-4. (canceled)

5. The composition of claim 1, wherein the semi-permanent colorant is genipin.

6. The composition of claim 1, wherein the solvent is present in an amount of about 65% to about 85% w/w of the composition.

7-8. (canceled)

9. The composition of claim 1, wherein the solvent comprises water, methanol, ethanol, isopropanol, diethylene glycol monoethyl ether, or a combination thereof.

10. The composition of claim 1, wherein the solvent is a combination of water and ethanol.

11. The composition of claim 10, wherein the water is present in an amount of about 25% to about 35% w/w of the composition, and/or wherein the ethanol is present in an amount of about 40% to about 55% w/w of the composition.

12-15. (canceled)

16. The composition of claim 1, wherein the film-forming agent is present in an amount of about 5% to about 10% w/w of the composition.

17-18. (canceled)

19. The composition of claim 1, wherein the film-forming agent comprises a volatile silicon resin, polyvinylpyrrolidone, an acrylate, an acrylamide, a copolymer, an isododecane resin, or a combination thereof.

20. The composition of claim 1, wherein the film-forming agent comprises polymethylsilsesquioxane, trimethylsiloxysilicate, polypropylsilsesquioxane, dimethicone, cyclopentasiloxane, dimethiconol crosspolymer, polysilicone-6, polysilicone-8, polysilicone-11, polysilicone-14, acrylates copolymer, styrene/acrylates copolymer, polyvinylpyrrolidone, acrylates/C12-22 alkyl methacrylate copolymer, acrylates/polytrimethylsiloxymethacrylate copolymer, polyvinylpyrrolidone/vinyl acetate (VP/VA) copolymer, VP/dimethiconylacrylate/polycarbamyl/polyglycol ester, VP/dimethylaminoethylmethacrylate copolymer, VP/dimethylamino ethylmethacrylate/polycarbamyl polyglycol ester, VP/eicosene copolymer, VP/hexadecene copolymer, VP/methacrylamide/vinyl imidazole copolymer, VP/polycarbamyl polyglycol ester, VP/VA copolymer, polyester-1, polyester-2, polyester-3, polyester-4, polyester-5, polyester-7, polyester-8, and polyester-10, or a combination thereof.

21. The composition of claim 1, wherein the film-forming agent is a combination of acrylates copolymer, polyvinylpyrrolidone, and styrene/acrylates copolymer.

22. The composition of claim 20, wherein the acrylates copolymer is present in an amount of about 0.1% to about 10% w/w of the composition, and/or wherein the polyvinylpyrrolidone is present in an amount of about 0.1% to about 10% w/w of the composition, and/or wherein the styrene/acrylates copolymer is present in an amount of about 0.1% to about 10% w/w of the composition.

23-30. (canceled)

31. The composition of claim 1, wherein the composition further comprises a humectant.

32-34. (canceled)

35. The composition of claim 31, wherein the humectant comprises Aloe vera gel, lithium chloride, allantoin, urea, dicyanamideglycerol, lactic acid, propylene glycol, polyethylene glycol, dipropylene glycol, polypropylene glycol, polyethylene glycol, propanediol, hexylene glycol, 1,3-butylene glycol, 1,2,6-hexanetriol, ethoxylated glycerol, propoxylated glycerol, glyceryl triacetate, sorbitol, hydroxypropyl sorbitol, xylitol, maltitol, hyaluronic acid, Tremella extract, sodium lactate, sodium L-pyroglutamate urea, and pyrrolidone carboxylic acid, or a combination thereof.

36. The composition of claim 31, wherein the humectant is propanediole.

37. The composition of claim 1, wherein the composition further comprises a preservative.

38-41. (canceled)

42. The composition of claim 37, wherein the preservative comprises a combination of phenoxyethanol and ethylhexylglycerin.

43. The composition of claim 42, wherein the combination of phenoxyethanol and ethylhexylglycerin is present in the composition at a ratio of about 9:1 phenoxyethanol:ethylhexylglycerin.

44. The composition of claim 42, wherein the combination of phenoxyethanol and ethylhexylglycerin is present in an amount of about 0.1% to about 2% w/w of the composition.

45. (canceled)

46. The composition of claim 37, wherein the preservative comprises EDTA.

47-48. (canceled)
